(19) United States
(12) Patent Application Publication
(10) Pub. No.: US 2008/0248187 A1
(43) Pub. Date: Oct. 9, 2008

(54) MIXTURE CONTAINING FATTY ACID GLYCERIDES

(76) Inventors: Ulrich Schoerken, Duesseldorf (DE); Peter Kempers, Moenchengladbach (DE); Andreas Sander, Illertissen (DE); Peter Horlacher, Bellenberg (DE)

Correspondence Address: SYNNESTVEDT & LECHNER LLP, 1101 MARKET STREET, PHILADELPHIA, PA, 19107, US

(21) Appl. No.: 12/061,276

(22) Filed: Apr. 2, 2008

(30) Foreign Application Priority Data
Apr. 2, 2007 (EP) 07006845.7
Apr. 2, 2007 (EP) 07006846.5

(51) Int. Cl. A23L 1/29; C12P 7/64; C07C 55/00

(52) U.S. Cl. 426/648; 554/227; 435/134

(57) ABSTRACT

A mixture containing fatty acid glycerides which has a high percentage content of PUFA acyl groups and a low percentage content of saturated fatty acid acyl groups is described. A process which enables the PUFA acyl groups in a mixture containing fatty acid glycerides (for example a fish oil) to be enriched and, at the same time, the content of saturated fatty acid acyl groups to be maintained at a low content is described. The process is a hydrolytic process or an alcoholysis in which the fatty acid acyl groups to be enriched are hydrolytically or alcoholytically released from the fatty acid glycerides slowly, if at all, the process being carried out in the presence of a lipase.

### CROSS-REFERENCE TO RELATED APPLICATIONS

This application claims priority from European application numbers EP 07006845.7 filed Apr. 2, 2007 and EP 07006846.5 filed Apr. 2, 2007, the entire contents of each of which are incorporated herein by reference.

### FIELD OF THE INVENTION

The present invention relates to a mixture containing fatty acid glycerides which has a high percentage content of PUFA acyl groups and a low percentage content of saturated fatty acid acyl groups. The invention also relates to a process which enables the PUFA acyl groups in a mixture containing fatty acid glycerides (for example, a fish oil) to be enriched and, at the same time, the content of saturated fatty acid acyl groups to be kept low. The process is a hydrolytic process or an alcoholysis in which the fatty acid acyl groups to be enriched are hydrolytically or alcoholytically released from the fatty acid glycerides only slowly, if at all, the process being carried out in the presence of a lipase.

### BACKGROUND OF THE INVENTION

In the context of the present invention, a fatty acid is a saturated or unsaturated, branched or unbranched aliphatic carboxylic acid. Fatty acids can be saturated, mono-unsaturated, di-unsaturated, tri-unsaturated, etc.

In the context of the present invention, fatty acids as such are also referred to as free fatty acids. By comparison, the expression “fatty acid acyl group” in the context of the present invention means the single-bonded residue which is obtained by removal of the H atom from the COOH group of a free fatty acid. Accordingly, fatty acid acyl groups occur, for example, in free fatty acids. They also occur in esters of fatty acids, for example esters with glycerol, the so-called glycerides. A fatty acid glyceride is an ester of glycerol and one, two or three fatty acid units. If only one OH group of the glycerol is esterified with a fatty acid unit, the ester is known as a monoglyceride. If two OH groups of the glycerol are each esterified with a fatty acid unit, the ester is known as a diglyceride. If all three OH groups of the glycerol are each esterified with a fatty acid unit, the ester is known as a triglyceride.

In the context of the present invention, PUFA means a fatty acid which is at least di-unsaturated. PUFA is the abbreviation for “polyunsaturated fatty acid”. In a preferred embodiment of the present invention, PUFA means an at least 5×-unsaturated fatty acid.

An omega-3 fatty acid in the context of the present invention is an at least di-unsaturated (and preferably at least 5×-unsaturated) fatty acid and is thus a PUFA. An omega-3 fatty acid has a double bond between the third and fourth carbon atoms counting from the methyl end, the methyl C atom being counted as the first C atom. Special omega-3 fatty acids are EPA ((all-Z)-5,8,11,14,17-eicosapentaenoic acid) and DHA ((all-Z)-4,7,10,13,16,19-docosahexaenoic acid).

Lipase A from Candida antarctica in the context of the present invention is the enzyme as described in J. Mol. Catal. B: Enzymatic 37 (2005), pages 36 to 46. Lipase A from Candida antarctica is commercially obtainable, for example as the liquid preparation Novozym® 735 from Novozymes A/S.

In the prior art, PUFA glycerides, i.e. glycerides in which PUFA acyl groups make up a large proportion of all the fatty acid acyl groups present, are produced in particular by one of the following two processes:
(1) Transesterification of fish oil to ethyl esters, enrichment of the PUFAs by distillation and re-synthesis to glycerides. The triglyceride synthesis is generally carried out enzymatically.
(2) Selective hydrolysis to fish oils to enrich the PUFAs in the glycerides and purification of the PUFA glyceride by distillation. The selective hydrolysis is generally carried out enzymatically.

Process (1), the enzymatic triglyceride synthesis, is described, for example, in EP-A 0 528 844.

Process (2) or the selectivity of lipases for PUFAs in the hydrolysis of glycerides is disclosed in several patent applications, including in particular WO 97/19601, WO 95/24459, WO 96/37586, WO 96/37587, EP-A 0 741 183, WO 96/26287, WO 00/73254, WO 04/043894, WO 00/49117 and WO 91/16443.

The following is known from the prior art on enzyme selectivities for PUFAs. Most lipases and phospholipases have a negative selectivity for PUFAs by comparison with other fatty acids typically present in vegetable and fish oils. By negative selectivity is meant that the lipases hydrolytically split off the other fatty acid groups from glycerides more quickly than the PUFA acyl groups. Accordingly, the enzymatic enrichment of PUFAs generally proceeds via a modification of the other “non-PUFA” fatty acids. This can be done by esterification, transesterification or hydrolysis of esters.

Negative selectivities for PUFAs are described, for example, for Candida and Mucor lipases. Some enzymes, for example those isolated from cold water fish, have a positive selectivity for PUFAs.

Lipase A from Candida antarctica is distinguished by some particular properties: high thermal stability, more an sn2- than an sn1,3-specificity (sn2- or sn1,3-specificity is the specificity of the lipase with respect to glycerides: sn1,3=preferred reaction at the external positions of glycerol, sn2=preferred reaction at the central position of glycerol), high selectivity for trans-fatty acids, a reaction with sterically hindered alcohols and a high chemoselectivity for N-acylation. A summary of the subject can be found in the following review article: J. Mol. Catal. B: Enzymatic 37 (2005), pages 36 to 46. A selectivity for saturated fatty acids is not described there.

Lipase preparations from Candida rugosa or from Candida cylindracea are a mixture of at least three enzymes, Lip 1, Lip 2 and Lip 3. Since commercial preparations are always mixtures of the individual lipases in variable proportions, characterization of the individual enzymes is difficult. Generally, it may be said that Lip 1 has a higher selectivity for linear alcohols while Lip 2 and Lip 3 are even capable of reacting sterically hindered alcohols. The reaction of tertiary alcohols has even been described for Candida rugosa lipase. A summary of the subject can be found in the following review article: Biotechnology Advances 24 (2006), pages 180 to 196.

Okada et al., Food Chemistry, Vol. 103, No. 4, 27 Mar., 2007, pages 1411-1419 disclose a glyceride mixture with a high content of at least tri-unsaturated fatty acid groups and a low content of saturated fatty acid groups. This mixture contains large amounts of EPA and DHA.

Yukihisa Tanaka et al., Journal of the American Oil Chemists Society, Vol. 69, No. 12, 1 Dec. 1992, pages 1210-1214 disclose the enrichment of DHA in fish oil by lipase-catalyzed hydrolysis. Lipase from Candida cylindraceae is used.

WO 88/02775 discloses the hydrolysis of olive oil, which contains polyunsaturated fatty acid residues, in the presence of lipase B from Candida antarctiabout.

WO 03/040091 discloses the hydrolysis of triglycerides in the presence of lipases.

Warwel, S. et al., Biotechnology Letters, Vol. 21, No. 5, 1999, pages 431-436 disclose the transesterification of fatty acid methyl esters with butanol in the presence of lipases.

WO 07/119,811 discloses the alcoholysis of PUFA-containing oils or fats in the presence of a lipase.

Fish oils consist essentially of triglycerides containing a mixture of saturated, mono- and poly-unsaturated fatty acids, more particularly with a high proportion of 5×- and 6×-unsaturated fatty acids, which may be used as a health-promoting food supplement. Since the highly unsaturated fatty acids in particular are health-promoting, there is an advantage in enriching them. This can be done, for example, by selective removal of the non-highly unsaturated fatty acids from the triglycerides, for example through selective enzymatic hydrolysis with lipases.

However, it should be noted in this regard that partial glycerides (1-mono-, 2-mono- and 1,3-diglycerides) have higher melting points than the corresponding triglyceride compounds (cf. Table 1).

TABLE 1
comparison of the melting points of various glycerides [° C.]

| Fatty | | | | | |
|---|---|---|---|---|---|
| acid | 1-Mono | 2-Mono | 1,2-Di | 1,3-Di | Tri |
| C8 | 40 | 30 | | | 8 |
| C10 | 53 | 40 | | 45 | 32 |
| C12 | 63 | 51 | 41 | 58 | |
| C14 | 71 | 61 | 56 | 66 | 58 |
| C16 | 77 | 69 | 63 | 73 | 66 |
| C18 | | 75 | 68 | 80 | 73 |
| C18:1 | 35 | | | 26 | 6 |
| C18:2 | 12 | | | −3 | −13 |

In Table 1 and in the following, the following standard abbreviation for fatty acids or fatty acid groups (acyl groups) is used: Cx:y denotes a fatty acid containing x carbon atoms and y double bonds.

It should also be noted that fish oils have a distinctly higher percentage content of saturated fatty acids than most vegetable oils (cf. Table 2).

TABLE 2
typical fatty acid compositions of various oils
Typical composition of the main constituents of food fats (1 = dairy fat;
2 = porcine tallow, 3 = bovine tallow, 4 = sunflower oil,
5 = soybean oil, 6 = olive oil, 7 = rapeseed oil, 8 = palm
oil, 9 = sardine oil, 10 = tuna oil, 11 = hydrogenated
vegetable oil (sunflower)) is described hereinafter.

| Fatty acid | 1 | 2 | 3 | 4 | 5 | 6 | 7 | 8 | 9 | 10 | 11 |
|---|---|---|---|---|---|---|---|---|---|---|---|
| C4:0 | 3 | | | | | | | | | | |
| C6:0-C10:0 | 5 | | | | | | | | | | |
| C12:0 | 3 | | | | | | | | | | |
| C14:0 | 10 | 3 | 4 | | | | 1 | 1 | 7 | 3 | |
| C16:0 | 33 | 24 | 30 | 4 | 10 | 11 | 4 | 39 | 18 | 21 | <10 |
| C16:1 | 4 | 3 | 3 | | | | | | 10 | 6 | |
| C18:0 | 9 | 8 | 22 | 4 | 4 | 2 | 1 | 5 | 3 | 6 | >90 |
| C18:1 | 26 | 46 | 38 | 23 | 22 | 75 | 59 | 45 | 15 | 19 | |
| C18:2 | 2 | 8 | 3 | 64 | 53 | 9 | 22 | 8 | | 2 | |
| C18:3 | | | 1 | | 8 | | 10 | | | 1 | |
| C20:5 | | | | | | | | | 17 | 7 | |
| C22:6 | | | | | | | | | 9 | 24 | |

If the saturated fatty acids in particular remain behind in the product mixture in the form of partial glycerides after partial hydrolysis, these glycerides can easily be precipitated in the enriched fish oil. Saturated partial glycerides have considerably higher melting points than unsaturated triglycerides. For example, 1-glycerol monopalmitate has a higher melting point of 77° C. than trioleate which has a melting point of 6° C.

If these saturated glycerides, particularly partial glycerides, remain in the product, the product loses its low-temperature stability. Flocculations occur in the product or the lipid mixture can even solidify.

The problem addressed by the present invention was to provide a mixture containing fatty acid glycerides, this mixture containing at least 5×-unsaturated fatty acid acyl groups and having a low melting point, so that it could readily be processed as a liquid without problems arising through crystallization of the mixture or parts of the mixture at low temperatures. The mixture would preferably have a high percentage content of at least 5×-unsaturated fatty acid acyl groups so that, for this reason, it would be particularly suitable for use as a food supplement or food additive.

### BRIEF SUMMARY OF THE INVENTION

The problem stated above is solved by the following mixture according to the invention.

The present invention relates to a mixture containing:

(A) optionally at least one monoglyceride corresponding to formula (I) or (II):

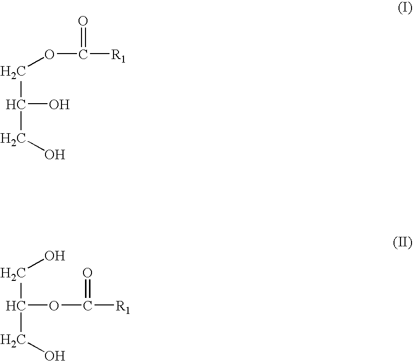

(B) optionally at least one diglyceride corresponding to formula (III) or (IV):

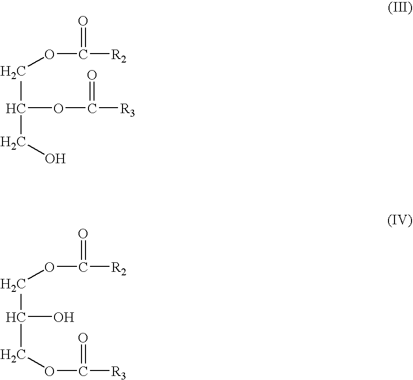

and
(C) at least one triglyceride corresponding to formula (V):

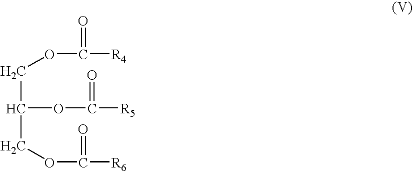

the groups R₁—CO—, R₂—CO—, R₃—CO—, R₄—CO—, R₅—CO— and R₆—CO— independently of one another being selected from the group consisting of a saturated fatty acid acyl group, a 1×- to 4×-unsaturated fatty acid acyl group and an at least 5×-unsaturated fatty acid acyl group,
the sum of the weights of all at least 5×-unsaturated fatty acid acyl groups present in the mixture, expressed as free fatty acids, based on the sum of the weights of all fatty acid acyl groups present in the mixture (again expressed as free fatty acids), amounting to at least 40% by weight and at most 80% by weight, more particularly to at least 45% by weight and at most 75% by weight and most particularly to at least 50% by weight and at most 70% by weight,
and the sum of the weights of all saturated fatty acid acyl groups present in the mixture (preferably the sum of the weights of the fatty acid acrylates of myristic acid (C14:0), palmitic acid (C16:0) and stearic acid (C18:0) present in the mixture), expressed as free fatty acid, based on the sum of the weights of all fatty acid acyl groups present in the mixture (again expressed as free fatty acids), amounting to at most

$$\frac{85 - a}{3} \% by weight ,$$

α being the sum of the weights of all at least 5×-unsaturated fatty acid acyl groups present in the mixture, expressed as free fatty acids, based on the sum of all fatty acid acyl groups present in the mixture (again expressed as free fatty acids) in % by weight,
and the weight of fatty acid acyl groups in stearic acid, expressed as free fatty acid, based on the sum of the weights of all fatty acid acyl groups present in the mixture (again expressed as free fatty acids), amounting to at most 2% by weight and preferably to at most 1.6% by weight,
and the content of triglycerides, based on all the glycerides corresponding to formulae I to V, being from 50 to 85% by weight and additionally meeting the requirement that the content of triglycerides, based on all the glycerides corresponding to formulae I to V, amounts to at least

$$\frac{410 - 5 \cdot a}{3} \% by weight ,$$

where α is as defined above,
and the content of diglycerides, based on all the glycerides corresponding to formulae I to V, being at least

100−3−b % by weight,

where b is the content of triglycerides, based on all the glycerides corresponding to formulae I to V, in % by weight.

The mixture according to the invention preferably contains at least one diglyceride, at least one triglyceride and, optionally, at least one monoglyceride.

The following mixtures are particular embodiments of the present invention.

The mixture according to the invention, this mixture additionally containing: at least 0.2% by weight of at least one component selected from the group consisting of a phospholipid, squalene and ceramide and/or at least 20 ppm of at least one component selected from the group consisting of vitamin A or provitamin A.

The mixture according to the invention or the mixture according to a particular embodiment already described, this mixture additionally containing free fatty acids and/or fatty acid ethyl esters, the sum of the weights of all free fatty acids or fatty acid ethyl esters present in the mixture amounting to at most 2% by weight, based on the sum of the weights of all monoglycerides plus the weights of all diglycerides plus the weights of all triglycerides plus the weights of all free fatty acids and/or fatty acid ethyl esters in the mixture.

The mixture according to the invention or the mixture according to a particular embodiment already described, the sum of the weights of all monoglycerides present in the mixture amounting to 0 to 3% by weight, based on the sum of the weights of all monoglycerides plus the weights of all diglycerides plus the weights of all triglycerides in the mixture.

The present invention also relates to the use of the mixture according to the invention or the mixture according to a particular embodiment already described as a food supplement or as a food additive for human nutrition.

A particular embodiment of the present invention is the above-described use as a feed for animal nutrition, more particularly for the aquaculture of marine organisms, more particularly fish and crustaceans.

The present invention also relates to a process for the production of the mixture according to the invention or the mixture according to any of the particular embodiments described above from a first mixture containing

(A) optionally at least one monoglyceride corresponding to formula (I) or (II):

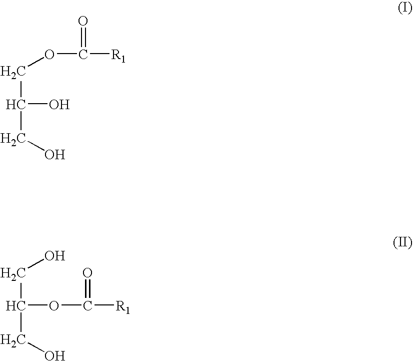

(B) optionally at least one diglyceride corresponding to formula (III) or (IV):

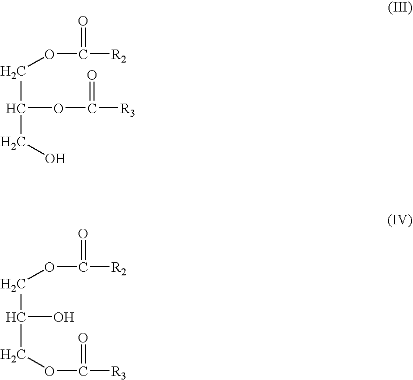

and
(C) at least one triglyceride corresponding to formula (V):

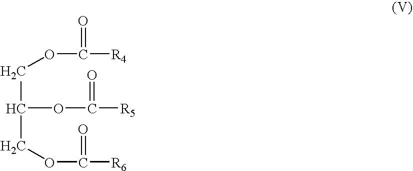

the groups R₁—CO—, R₂—CO—, R₃—CO—, R₄—CO—, R₅—CO— and R₆—CO— independently of one another being selected from the group consisting of a saturated fatty acid acyl group, a 1×- to 4×-unsaturated fatty acid acyl group and an at least 5×-unsaturated fatty acid acyl group,
and the sum of the weights of all at least 5×-unsaturated fatty acid acyl groups present in the mixture, expressed as free fatty acids, based on the sum of the weights of all fatty acid acyl groups present in the mixture (again expressed as free fatty acids), being higher than in the first mixture by a factor of at least 1.3 (factor A),
and the sum of the weights of all saturated fatty acid acyl groups present in the mixture (preferably the sum of the weights of the fatty acid acrylates of myristic acid (C14:0), palmitic acid (C16:0) and stearic acid (C18:0) present in the mixture), expressed as free fatty acid, based on the sum of the weights of all fatty acid acyl groups present in the mixture (again expressed as free fatty acids), being lower than in the first mixture by a factor of at least 2 (factor B),
and the value of factor A being smaller than the value of factor B,
and the content of triglycerides in the first mixture, based on all the glycerides corresponding to formula I to V, being at least 90% by weight,
and the first mixture containing at least 5×-unsaturated fatty acid acyl groups and the sum of the weights of all at least 5×-unsaturated fatty acid acyl groups present in the first mixture, expressed as free fatty acids, based on the sum of the weights of all fatty acid acyl groups present in the first mixture (again expressed as free fatty acids), in a preferred embodiment of the invention being at least 15% by weight,
and the process comprising reacting the first mixture with water (hydrolysis) or with a C₁₋₄ alcohol (alcoholysis) in the presence of a first, non-regioselective lipase with a high specificity for saturated fatty acids by comparison with all unsaturated fatty acids (preferably lipase A from Candida antarctica).

### BRIEF DESCRIPTION OF THE SEVERAL VIEWS OF THE DRAWINGS

FIG. 1: shows the degrees of hydrolysis of mixtures 1 and 2 graphically compared with mixture 3 which contains the enzyme mixture for the same overall enzyme activity (Crug=Candida rugosa lipase (mixture 2), Cant=Candida antarctica A lipase (mixture 1) and Mixed=mixture of both lipases (mixture 3)).

FIG. 2: shows the degrees of hydrolysis of mixtures 1 and 4 graphically compared with mixture 5 which contains the enzyme mixture for the same overall enzyme activity (Ccyl=Candida cylindracea lipase (mixture 4), Cant=Candida antarctica A lipase (mixture 1) and Mixed=mixture of both lipases (mixture 5)).

FIG. 3: shows the degrees of hydrolysis of mixtures 1 and 2 graphically compared with mixture 3 which contains the enzyme mixture for the same overall enzyme activity (Crug=Candida rugosa lipase (mixture 2), Cant=Candida antarctica A lipase (mixture 1) and Mixed=mixture of both lipases (mixture 3)).

FIG. 4: shows the degrees of hydrolysis of mixtures 1 and 4 graphically compared with mixture 5 which contains the enzyme mixture for the same overall enzyme activity (Ccyl=Candida cylindracea lipase (mixture 4), Cant=Candida antarctica A lipase (mixture 1) and Mixed=mixture of both lipases (mixture 5)).

FIG. 5: shows comparative plotting of the omega-3 fatty acids of the individual mixtures in the glyceride and fatty acid fraction (Cant hydrolyzate=fatty acids released from mixture 1; Cant glyceride=glyceride-bound fatty acids from mixture 1, Crug hydrolyzate=fatty acids released from mixture 2; Crug glyceride=glyceride-bound fatty acids from mixture 2, mixed hydrolyzate=fatty acids released from mixture 3; mixed glyceride=glyceride-bound fatty acids from mixture 3).

FIG. 6: shows comparative plotting of the saturated fatty acids of the individual mixtures in the glyceride and fatty acid fraction (Cant hydrolyzate=fatty acids released from mixture 1; Cant glyceride=glyceride-bound fatty acids from mixture 1, Crug hydrolyzate=fatty acids released from mixture 2; Crug glyceride=glyceride-bound fatty acids from mixture 2, mixed hydrolyzate=fatty acids released from mixture 3; mixed glyceride=glyceride-bound fatty acids from mixture 3).

FIG. 7: shows comparative plotting of the saturated and unsaturated C14-C18 fatty acids of the individual mixtures in the glyceride and fatty acid fraction (Cant hydrolyzate=fatty acids released from mixture 1; Cant glyceride=glyceride-bound fatty acids from mixture 1, Crug hydrolyzate=fatty acids released from mixture 2; Crug glyceride=glyceride-bound fatty acids from mixture 2, mixed hydrolyzate=fatty acids released from mixture 3; mixed glyceride=glyceride-bound fatty acids from mixture 3).

FIG. 8: shows comparative plotting of the omega-3 fatty acids of the individual mixtures in the glyceride and fatty acid fraction (Cant hydrolyzate=fatty acids released from mixture 1; Cant glyceride=glyceride-bound fatty acids from mixture 1, Ccyl hydrolyzate=fatty acids released from mixture 4; Ccyl glyceride=glyceride-bound fatty acids from mixture 4, mixed hydrolyzate=fatty acids released from mixture 5; mixed glyceride=glyceride-bound fatty acids from mixture 5).

FIG. 9: shows comparative plotting of the saturated fatty acids of the individual mixtures in the glyceride and fatty acid fraction (Cant hydrolyzate=fatty acids released from mixture 1; Cant glyceride=glyceride-bound fatty acids from mixture 1, Ccyl hydrolyzate=fatty acids released from mixture 4; Ccyl glyceride=glyceride-bound fatty acids from mixture 4, mixed hydrolyzate=fatty acids released from mixture 5; mixed glyceride=glyceride-bound fatty acids from mixture 5).

FIG. 10: shows comparative plotting of the saturated and unsaturated C14-C18 fatty acids of the individual mixtures in the glyceride and fatty acid fraction (Cant hydrolyzate=fatty acids released from mixture 1; Cant glyceride=glyceride-bound fatty acids from mixture 1, Ccyl hydrolyzate=fatty acids released from mixture 4; Ccyl glyceride=glyceride-bound fatty acids from mixture 4, mixed hydrolyzate=fatty acids released from mixture 5; mixed glyceride=glyceride-bound fatty acids from mixture 5).

FIG. 11: shows a graphic representation of the maximal and semi-maximal contents of 5×- and 6×-unsaturated fatty acids and the corresponding contents of saturated fatty acids bound in the glyceride fraction for a conversion of 0 to 70% calculated for the fish oil Napro 18/12.

FIG. 12: shows a graphic representation of the maximal and semi-maximal contents of 5×- and 6×-unsaturated fatty acids and the corresponding contents of saturated fatty acids bound in the glyceride fraction for a conversion of 0 to 70% calculated for the fish oil Napro 11/19.

FIG. 13: shows a graphic representation of the maximal and semi-maximal contents of 5×- and 6×-unsaturated fatty acids and the corresponding contents of saturated fatty acids bound in the glyceride fraction for a conversion of 0 to 70% calculated for the fish oil Lamotte Type 170.

FIG. 14: shows a graphic representation of the maximal and semi-maximal contents of 5×- and 6×-unsaturated fatty acids and the corresponding contents of saturated fatty acids bound in the glyceride fraction for a conversion of 0 to 70% calculated for the fish oil Brudy Algatrium DHA 18.

### DETAILED DESCRIPTION OF THE INVENTION

The following variants are particular embodiments of the invention.

The process according to the invention, the reaction of the first mixture with water (hydrolysis) or with an alcohol containing 1 to 4 carbon atoms (alcoholysis) being carried out in the presence of a first, non-regioselective lipase with a high specificity for monounsaturated fatty by comparison with all unsaturated fatty acids.

The process according to the invention or the process according to a particular embodiment already described, the first lipase being lipase A from Candida antarctica or an at least 80% structurally similar lipase.

The process according to the invention or the process according to a particular embodiment already described, the second lipase being selected from one of the lipases from Candida rugosa or Candida cylindracea or an at least 80% structurally similar lipase and the second lipase preferably being a lipase from Candida rugosa or a lipase from Candida cylindracea or a mixture of these two lipases.

The process according to the invention or the process according to a particular embodiment already described, the first mixture being selected from the group consisting of a fish oil, an oil from marine crustaceans (more particularly krill oil), an oil from microalgae, an oil from marine microorganisms (more particularly from protists) and an oil from marine mammals, an oil containing more than 20% by weight and, more particularly, more than 30% by weight bound at least 5×-unsaturated fatty acids preferably being used.

The process according to the invention or the process according to a particular embodiment already described, the first lipase being used in free or immobilized form, preferably in immobilized form adsorbed onto nonionic polymers, more particularly onto polypropylene or a polyacrylate-based carrier material.

The process according to the invention or the process according to a particular embodiment already described, the second lipase being used in free or immobilized form.

The process according to the invention or the process according to a particular embodiment already described, water being added in a concentration of 2 to 50% by weight for partial hydrolysis or a short-chain alcohol, preferably ethanol, being added in a concentration of 5 to 50% by weight or a mixture of water and alcohol with a total concentration of 5 to 50%, based on the oil component, being added.

The process according to the invention or the process according to a particular embodiment already described, the reaction being carried out as a batch reaction with stirring at a temperature of 20 to 80° C.

The process according to the invention or the process according to a particular embodiment already described, the reaction being carried out as a continuous reaction with stirring at a temperature of 20 to 80° C. with the aqueous or alcoholic phase in co-current or in counter-current.

The process according to the invention or the process according to a particular embodiment already described, this process additionally comprising: removing the water- and/or alcohol-containing phase by separation, optionally removal of immobilized enzyme by filtration and removal of the fatty acids or fatty acid esters released by molecular distillation, in which the mixture to be produced remains behind as the residue. A particular embodiment is this process, the distillation being carried out at a temperature below 210° C. and under a pressure of at most 0.3 mbar.

The process according to the invention or the process according to a particular embodiment already described, this process additionally comprising: removal of the water- and/or alcohol-containing phase by separation, optionally removal of immobilized enzyme by filtration and a first removal of the fatty acids or fatty acid esters with a chain length <20 released by molecular distillation and a second removal of long-chain fatty acids or fatty acid esters with a chain length of >C20 and monoglycerides by molecular distillation, in which the mixture to be produced remains behind as the residue and a monoglyceride-rich product is obtained as the distillate of the second molecular distillation. A particular embodiment is this process, the first distillation being carried out at a temperature below 180° C. and under a pressure of at most 0.3 mbar and the second distillation being carried out at a temperature below 210° C. and under a pressure of at most 0.3 mbar.

The process according to the invention or the process according to a particular embodiment already described the mixture to be produced being purified by bleaching, more particularly by treatment with active carbon.

The process according to the invention or the process according to a particular embodiment already described, the mixture to be produced being purified by deodorization, more particularly by deodorization with steam or nitrogen, at a temperature below 200° C. either in batches or continuously.

The process according to the invention or the process according to a particular embodiment already described, the monoglyceride-rich product being used for enzymatic re-synthesis with immobilized lipase B from Candida antarctica at a temperature of 45 to 80° C. and under a pressure of less than 30 mbar.

The process according to the invention or the process according to a particular embodiment already described, the glyceride-rich mixture being converted into the corresponding ethyl ester by alkaline transesterification with ethanol and an ethylate salt as catalyst at temperatures of 40 to 120° C.

The process according to the invention or the process according to a particular embodiment already described, the ethyl ester being split by fractional distillation, preferably by molecular distillation, at temperatures below 180° C. into a fraction enriched with 5×- and 6×-unsaturated fatty acids and a depleted fraction.

Lipase A from Candida antarctica has the following advantages:
1) negative selectivity for highly unsaturated fatty acids (PUFAs, more particularly for EPA and DHA) and
2) positive selectivity for saturated fatty acids (more particularly for myristic acid, palmitic acid and stearic acid).

It is thus possible to provide enriched glycerides with a high PUFA content and a low content of saturated components and, more particularly, a low content of saturated partial glycerides.

Another advantage of the present invention is that it provides a hydrolysis and working-up process, in which a glyceride mixture with an increased PUFA concentration and a reduced concentration of saturated fatty acids can be obtained. The percentage content of free acids in the glyceride mixture can be <1% and the content of monoglycerides <5%. The low content of free fatty acids and monoglycerides is important because these compounds have negative sensory properties (bitter taste) compared with di- and triglycerides and, in the form of saturated fatty acids, easily lead to product clouding.

It has been found that lipase A from Candida antarctica has a distinctly negative selectivity for PUFAs and a positive selectivity for saturated fatty acids. No other commercially obtainable enzyme with pronounced positive selectivity for saturated fatty acids was found. The positive selectivity for lipase A from Candida antarctica has not hitherto been described and only a selectivity of the enzyme for unsaturated fatty acids with a double bond in the trans position is known from the prior art.

A process for the production of enriched PUFA glycerides has been developed. In this process, a mixture of fish oil and water is stirred in the presence of lipase A from Candida antarctica and the degree of hydrolysis is monitored via the formation of the free acid. The lipase may be used in liquid or immobilized form. After the desired degree of hydrolysis has been reached, the water and the enzyme are removed. The glyceride product is dried and the free fatty acids are removed from the glyceride mixture by molecular distillation. This may optionally be followed by bleaching and/or deodorization of the product by standard methods.

The glyceride mixtures produced in accordance with the invention show a distinct enrichment of the PUFAs and a depletion of the saturated fatty acids, based on the starting composition. The glyceride mixtures thus obtained have better low-temperature stability than the enriched PUFA glycerides which were produced with a negatively PUFA-selective, but non-selective enzyme for the saturated fatty acids (for example Candida rugosa or Candida cylindracea lipase).

The combination of lipase A from Candida antarctica and Candida cylindracea lipase in the hydrolysis step also leads to a PUFA glyceride with a depleted content of saturated fatty acids.

Lipase A from Candida antarctica is commercially obtainable, for example under the name of Novozym® 735 from Novozymes A/S, Bagsvaerd, Denmark.

Lipase from Candida cylindracea is commercially obtainable, for example under the name of Lipomod® 34 from Biocatalysts Ltd., Pontypridd, UK.

The other lipases used in the following Examples are also commercially obtainable. The lipase from Geotrichum candidum was self-produced.

In the following, U stands for “unit” and is an indication of the activity of enzymes. 1 U is the reaction of 1 μmol substance per minute under certain, defined reaction conditions.

Determining the activity of lipase A from Candida antarctica is carried out as follows (for Novozymes A/S by the method for Novozym® 735): the release of butyric acid from glycerol tributyrate is determined at 30° C./pH 7. A 0.16 M tributyrin solution is used for this purpose and butyric acid is titrated with NaOH at a constant pH. 1 unit corresponds to the activity which releases 1 μmol butyric acid per minute from tributyrin.

Determining the activity of lipase from Candida cylindracea is carried out as follows (by the Biocatalysts Ltd. method for Lipomod® 34): the release of fatty acid from olive oil in an aqueous emulsion is determined at a constant pH by NaOH titration. 1 unit corresponds to the activity which releases 1 μmol fatty acid per minute from olive oil.

Determining the activity of lipase from Candida rugosa is carried out as follows (by the Amano Inc. method for Lipase AY): the release of fatty acid from triglycerides in an aqueous triglyceride emulsion is determined at a constant pH by NaOH titration. 1 unit corresponds to the activity which releases 1 μmol fatty acid per minute from triglyceride.

In the process according to the invention, lipase A from Candida antarctica may be used on its own or in combination with a second lipase having negative selectivity for PUFAs preferably selected from the group of Candida lipases.

The lipase may be used in free or immobilized form.

Free form means, for example, that the lipase is directly dissolved in the aqueous part of the reaction mixture.

Immobilized form means, for example, immobilization on adsorber resins or porous plastics and immobilization on ion exchangers. Alternatively, the lipase may be immobilized on a carrier through covalent bonds. Adsorber resins (for example polystyrenes or polyacrylate types) and porous plastics (for example polypropylene) are preferred.

A second lipase may be used either at the same time as lipase A from Candida antarctica or at a later time as a second enzyme for increasing the degree of hydrolysis. The second enzyme is preferably used at a later time for increasing the degree of hydrolysis.

Particular embodiments of the process according to the invention are described in the following:
Suitable oils as starting materials: all fish oils are suitable, as are oils from PUFA-producing microorganisms (generally algae or fungi), marine mammals and lower marine animals (for example krill). Fish oils and microbial oils are preferred. Purified oils or crude oils may be used.
Reaction temperature: 20-80° C., preferably 30-60° C.
Percentage water content of the mixture as a whole: 2-80%, preferably 5-50%
Other constituents for the enzymatic reaction: none necessary
Quantity of lipase A from Candida antarctica: may be freely selected according to the desired reaction time and degree of hydrolysis. Typical values: 5-500 U free lipase/g fish oil. 1-10% by weight immobilized lipase with a bound lipase content corresponding to 500 to 10,000 U free lipase per g carrier
2nd enzyme: preferably Candida cylindracea or Candida rugosa lipase. Typical quantities of enzyme: 5-500 U free lipase/g fish oil. 1-10% by weight immobilized lipase with a bound lipase content corresponding to 500 to 10,000 U free lipase per g carrier
Reaction time: dependent on the desired degree of hydrolysis and the quantity of enzyme used. Typical values: 4-100 h.
Degree of hydrolysis: can be freely selected. Suitable range 15-70%, preferred range 25-60%.

Particular embodiments of the process according to the invention in relation to working up of the product are described in the following:
Removal of the fatty acids by distillation. Typical distillation parameters: vacuum<1 mbar, T>/=150° C.
Removal of monoglycerides by distillation. Typical distillation parameters: vacuum<1 mbar, T>/=190° C.
Refining to remove residual fatty acids
Bleaching of the product (for example with bleaching earths, active carbons)
Deodorization of the product (for example deodorization with steam or nitrogen, treatment with active carbon)
Stabilization of the product with antioxidants

Particular embodiments of the mixture according to the invention are described in the following:

Sum of EPA+DHA: >35%, preferably >40%

Sum of PUFAs: >40%, preferably >50%

Sum of omega-3 fatty acids: >40%, preferably >45%

Percentage content of free acid: <10%, preferably <2%

Percentage content of monoglycerides: <15%, preferably <5%

Percentage content of di- and triglycerides: >80%, preferably >90%

Ratio of diglycerides to triglycerides: 1:100 to 50:50

Percentage content of bound fatty acids: <25%, preferably <20%

The combination of lipase A from Candida antarctica and another lipase from the genus Candida (preferably either lipase from Candida rugosa or lipase from Candida cylindracea or a mixture of these two lipases) has the following advantages:
3) Negative selectivity for highly unsaturated fatty acids (PUFAs, more particularly for EPA and DHA) and
4) Positive selectivity for saturated fatty acids (more particularly for myristic acid, palmitic acid and stearic acid).
5) Synergistic effect in the selective hydrolysis of fish oils, characterized by
   a higher hydrolysis rate than the particular enzymes on their own for the same enzyme input based on activity
   a higher attainable degree of hydrolysis per unit of time. Mixtures with a very high PUFA content inter alia can be obtained in this way. By virtue of the high degree of hydrolysis then achieved, these mixtures often have a high percentage content of diglycerides, occasionally even more diglycerides than triglycerides
   equally good negative selectivity for omega-3 fatty acids as the respective enzymes on their own.

The invention thus provides enriched glycerides with a high PUFA content and a low content of saturated components, more particularly a low percentage content of saturated partial glycerides.

It has been found that the simultaneous use of Candida antarctica A lipase with a second lipase from the genus Candida (Candida rugosa or Candida cylindracea lipase) has a synergistic hydrolytic effect. A higher hydrolysis rate is achieved and a higher concentration of omega-3 fatty acids can be obtained in the glyceride fraction for the same quantity of enzyme.

The following examples are illustrative of the invention and should not be construed in any manner as limiting the scope of the invention.

### EXAMPLES

### Example 1

### Screening for Positive Selectivity for Saturated Fatty Acids on the Basis of Thistle Oil

10 g thistle oil and 5 g water were incubated with various enzymes while stirring at room temperature. Samples of the oil phase were then analyzed for the composition of the fatty acids released. The sum of palmitic and stearic acid in the starting oil was 10.0%.

TABLE 3
enzyme screening on the basis of thistle oil

| Lipase from organism | Sum of palmitic and stearic acid |
|---|---|
| Alcaligenes sp | 17.5% |
| Aspergillus niger | 23.3% |
| Aspergillus oryzae | 22.7% |
| Candida antarctica A | 29.0% |
| Candida cylindracea | 7.6% |
| Candida rugosa | 8.8% |
| Geotrichum candidum | 3.6% |
| Penicilium roqueforti | 15.4% |
| Pseudomonas fluorescens | 18.3% |
| Pseudomonas sp. | 11.2% |
| Rhizopus javanicus | 18.9% |
| Rhizopus niveus | 17.7% |
| Rhizopus oryzae | 19.2% |
| Thermomyces lanugenosus | 22.9% |

Most enzymes showed an enrichment of saturated fatty acids in the hydrolyzate which is generally attributable to a negative selectivity for trilinoleate. Lipase A from Candida antarctica had the highest percentage content of saturated fatty acids in the hydrolyzate.

### Example 2

### Screening for Positive Selectivity for Saturated Fatty Acids on the Basis of Rapeseed Oil

10 g rapeseed oil and 5 g water were incubated with various enzymes while stirring at room temperature. Samples of the oil phase were then analyzed for the composition of the fatty acids released. The sum of palmitic and stearic acid in the starting oil was 6.5%.

TABLE 4
enzyme screening on the basis of rapeseed oil

| | Sum of palmitic |
|---|---|
| Lipase from organism | and stearic acid |
| Aspergillus niger | 8.6% |
| Aspergillus oryzae | 7.2% |
| Candida antarctica A | 10.3% |
| Candida cylindracea | 5.8% |
| Candida rugosa | 5.6% |
| Geotrichum candidum | 1.7% |
| Mucor javanicus | 8.9% |
| Penicilium roqueforti | 6.9% |
| Porcine pancreas | 8.1% |
| Pseudomonas cepacia | 8.0% |
| Pseudomonas fluorescens | 7.7% |
| Pseudomonas stutzeri | 7.4% |
| Rhizomucor miehei | 7.8% |
| Rhizopus javanicus | 8.4% |
| Rhizopus niveus | 8.8% |
| Rhizopus oryzae | 8.1% |
| Thermomyces lanugenosus | 8.1% |

Some of the enzymes showed a slight enrichment of saturated fatty acids in the hydrolyzate which is distinctly less pronounced than in the hydrolysis of thistle oil. In this case, too, lipase A from Candida antarctica had the highest percentage content of saturated fatty acids in the hydrolyzate.

### Example 3

### Screening for Negative Selectivity for PUFAs on the Basis of Mackerel Oil

10 g mackerel oil and 10 g water were introduced into a flask and heated with stirring to 45° C. or 60° C. Various enzymes were then added in a quantity of at most 1% free enzyme (commercial enzyme preparation) or 3% immobilized enzyme, after which the mixtures were incubated with stirring. Samples were taken during the reaction and the conversion was determined by measurement of the acid value. From a conversion of >40% degree of hydrolysis, the fatty acid composition of the fatty acids released was analyzed. The content of glyceride-bound omega-3 fatty acids (mainly EPA and DHA present) was calculated from these data. Enzymes which did not achieve a conversion of 40% over a reaction time of 24 h were not further evaluated. The content of omega-3 fatty acids in the starting oil was 37.6%.

TABLE 5
enzyme screening on the basis of mackerel oil

| | | | | Omega-3 |
|---|---|---|---|---|
| Enzyme | Form | Temperature | Conversion | in glyceride |
| Alcaligenes sp. | Free | 45° C. | 62% | 51% |
| Aspergillus niger | Free | 45° C. | <40% | |
| Candida antarctica A | Free | 45° C. | 44% | 52% |
| Candida antarctica A | Immobilized | 60° C. | <40% | |
| Candida antarctica B | Free | 45° C. | <40% | |
| Candida antarctica B | Immobilized | 60° C. | 43% | 41% |
| Candida cylindracea | Free | 45° C. | 43% | 55% |
| Candida rugosa | Free | 45° C. | 41% | 55% |
| Pseudomonas fluorescens | Free | 45° C. | 45% | 47% |
| Rhizomucor miehei | Immobilized | 60° C. | <40% | |
| Rhizopus oryzae | Free | 45° C. | 43% | 43% |
| Rhizopus niveus | Free | 45° C. | <40% | |
| Thermomyces lanugenosus | Free | 45° C. | 61% | 48% |
| Thermomyces lanugenosus | Immobilized | 45° C. | 60% | 51% |

Coupled with good hydrolysis activity, the lipases from the organism Candida (except for lipase B from Candida antarctica) and Alcaligenes above all showed good negative selectivity for PUFAs. All the enzymes tested showed slight negative selectivity for PUFAs.

### Example 4

### Screening for Negative Selectivity for PUFAs on the Basis of Menhaden Oil

10 g menhaden oil and 10 g water were introduced into a flask and heated with stirring to 45° C. or 60° C. Various enzymes were then added in a quantity of at most 1% free enzyme (commercial enzyme preparation) or 3% immobilized enzyme, after which the mixtures were incubated with stirring. Samples were taken during the reaction and the conversion was determined by measurement of the acid value. From a conversion of >40% degree of hydrolysis, the fatty acid composition of the fatty acids released was analyzed. The content of glyceride-bound omega-3 fatty acids (mainly EPA and DHA present) was calculated from these data. Enzymes which did not achieve a conversion of 40% over a reaction time of 24 h were not further evaluated. The content of omega-3 fatty acids in the starting oil was 38.0%.

TABLE 6
enzyme screening on the basis of menhaden oil

| | | | | Omega-3 in |
|---|---|---|---|---|
| Enzyme | Form | Temperature | Conversion | glyceride |
| Alcaligenes sp. | Free | 45° C. | 58% | 53% |
| Aspergillus niger | Free | 45° C. | <40% | |
| Candida antarctica A | Free | 45° C. | 52% | 59% |
| Candida antarctica A | Immobilized | 60° C. | <47% | 56% |
| Candida antarctica B | Free | 45° C. | <40% | |
| Candida antarctica B | Immobilized | 60° C. | 43% | 45% |
| Candida cylindracea | Free | 45° C. | 49% | 61% |
| Candida rugosa | Free | 45° C. | <40% | |
| Pseudomonas fluorescens | Free | 45° C. | 47% | 49% |
| Rhizomucor miehei | Immobilized | 60° C. | <40% | |
| Rhizopus oryzae | Free | 45° C. | <40% | |
| Rhizopus niveus | Free | 45° C. | <40% | |
| Thermomyces lanugenosus | Free | 45° C. | 41% | 49% |
| Thermomyces lanugenosus | Immobilized | 45° C. | 42% | 50% |

Coupled with good hydrolysis activity, the lipases from the organism Candida (except for lipase B from Candida antarctica) and Alcaligenes above all showed good negative selectivity for PUFAs. All the enzymes tested showed slight negative selectivity for PUFAs.

### Example 5

### Screening for Negative Selectivity for PUFAs on the Basis of Tuna Oil

10 g tuna oil and 10 g water were introduced into a flask and heated with stirring to 45° C. or 60° C. Various enzymes were then added in a quantity of at most 1% free enzyme (commercial enzyme preparation) or 3% immobilized enzyme, after which the mixtures were incubated with stirring. Samples were taken during the reaction and the conversion was determined by measurement of the acid value. From a conversion of >40% degree of hydrolysis, the fatty acid composition of the fatty acids released was analyzed. The content of glyceride-bound omega-3 fatty acids (mainly EPA and DHA present) was calculated from these data. Enzymes which did not achieve a conversion of 40% over a reaction time of 24 h were not further evaluated. The content of omega-3 fatty acids in the starting oil was 39.6%.

TABLE 7
enzyme screening on the basis of tuna oil

| | | | | Omega-3 in |
|---|---|---|---|---|
| Enzyme | Form | Temperature | Conversion | glyceride |
| Alcaligenes sp. | Free | 45° C. | 58% | 53% |
| Aspergillus niger | Free | 45° C. | <40% | |
| Candida antarctica A | Free | 45° C. | 63% | 64% |
| Candida antarctica A | Immobilized | 60° C. | <40% | |
| Candida antarctica B | Free | 45° C. | <40% | |
| Candida antarctica B | Immobilized | 60° C. | 43% | 46% |
| Candida cylindracea | Free | 45° C. | 53% | 64% |
| Candida rugosa | Free | 45° C. | 41% | 57% |
| Pseudomonas fluorescens | Free | 45° C. | 46% | 50% |
| Rhizomucor miehei | Immobilized | 60° C. | <40% | |
| Rhizopus oryzae | Free | 45° C. | 54% | 49% |
| Rhizopus niveus | Free | 45° C. | <40% | |
| Thermomyces lanugenosus | Free | 45° C. | 61% | 54% |
| Thermomyces lanugenosus | Immobilized | 45° C. | 55% | 48% |

Coupled with good hydrolysis activity, lipases from the organism Candida (except for lipase B from Candida antarctica) above all showed good negative selectivity for PUFAs. All the enzymes tested showed slight negative selectivity for PUFAs. This selectivity is more pronounced with DHA-rich tuna oil than with the EPA-rich fish oils.

### Example 6

### Selective Hydrolysis of Fish Oil 18/12 with Lipase A from Candida Antarctica

### A) Enzyme Immobilization

400 g acetone were added to 40 g Accurel® MP 1000 (a porous polypropylene powder obtainable from Membrana GmbH, Obernburg, Germany), followed by stirring for 5 minutes. The acetone was filtered off and the Accurel was washed with water. 800 g water and 40 g of a commercially obtainable lipase A from Candida antarctica (Novozym® 735) were added to the Accurel. The mixture was incubated for 20 h and then filtered off. The immobilizate was washed with water and used moist for the hydrolysis.

### B) Reaction+Working Up

800 g fish oil 18/12 (a fish oil with the composition shown in the following Table) and 800 g water were introduced into a double-jacketed reactor and heated with stirring to 45° C. The moist enzyme immobilizate was added to the mixture which was then incubated with stirring at a constant temperature. The acid value was measured hourly. At an acid value of about 60, the stirrer was switched off. After phase separation, the aqueous phase was removed and the oil phase was dried at 80° C. in a rotary evaporator. The acid value after separation and drying was 67. The oil phase was worked up by short-path distillation. The free acids were distilled overhead at a temperature of 200° C. and a vacuum of 0.3 mbar. The bottom product is the enriched PUFA glyceride.

### C) Product Analysis

The product and, for comparison, the starting material were analyzed by GPC (gel permeation chromatography) (glycide distribution); GC (gas chromatography) (fatty acid sample after methylation) and the acid value was measured. The results are set out in Table 8.

TABLE 8
fatty acid distribution and composition in the glyceride and in the
starting product

| | | PUFA |
|---|---|---|
| Fatty acid | Fish oil 18/12 [%] | glyceride [%] |
| 14:0 | 6.7 | 3.2 |
| 16:0 | 16.6 | 7.4 |
| 16:1 | 8.0 | 7.5 |
| 16:4 | 2.1 | 2.6 |
| 18:0 | 3.4 | 1.6 |
| 18:1 | 12.2 | 11.3 |
| 18:2 | 1.3 | 1.3 |
| 18:3 | 0.9 | 0.8 |
| 18:4 | 3.3 | 4.0 |
| 20:1 | 1.5 | 1.4 |
| 20:4 | 1.1 | 1.5 |
| 20:5 | 18.4 | 24.2 |
| 22:5 | 2.2 | 2.7 |
| 22:6 | 12.8 | 18.5 |
| Acid value | | 0.7 |
| Triglyceride | >95 | 72.6 |
| Diglyceride | | 27.1 |
| Monoglyceride | | 0.4 |
| Fatty acid | | <0.5 |

### Example 7

### Selective Hydrolysis of Fish Oil 18/12 with Lipase a from Candida Cylindracea

A) Reaction+working up

1,000 g fish oil 18/12 (a fish oil with the composition shown in the following Table) and 250 g water were introduced into a double-jacketed reactor and heated with stirring to 45° C. 750 mg of a commercially obtainable lipase from Candida cylindracea (Lipomod® 34) were added to the mixture which was then incubated with stirring at a constant temperature. The acid value was measured hourly. At an acid value of about 60, the stirrer was switched off. After phase separation, the aqueous phase was removed and the oil phase was dried at 80° C. in a rotary evaporator. The acid value after separation and drying was 60. The oil phase was worked up by short-path distillation. The free acids were distilled overhead at a temperature of 200° C. and a vacuum of 0.3 mbar. The sump product is the enriched PUFA glyceride.

### C) Product Analysis

The product and, for comparison, the starting material were analyzed by GPC (glycide distribution); GC (fatty acid sample after methylation) and the acid value was measured. The results are set out in Table 9.

TABLE 9
fatty acid distribution and composition in the glyceride and in the
starting product

| | Fish oil | |
|---|---|---|
| Fatty acid | 18/12 [%] | PUFA glyceride [%] |
| 14:0 | 6.7 | 6.0 |
| 16:0 | 16.6 | 13.2 |
| 16:1 | 8.0 | 3.8 |
| 16:4 | 2.1 | 2.5 |
| 18:0 | 3.4 | 3.4 |
| 18:1 | 12.2 | 7.4 |
| 18:2 | 1.3 | 0.7 |
| 18:3 | 0.9 | 0.5 |
| 18:4 | 3.3 | 4.1 |
| 20:1 | 1.5 | 1.7 |
| 20:4 | 1.1 | 1.4 |
| 20:5 | 18.4 | 22.6 |
| 22:5 | 2.2 | 2.9 |
| 22:6 | 12.8 | 18.0 |
| Acid value | | 1.4 |
| Triglyceride | >95 | 78.0 |
| Diglyceride | | 20.5 |
| Monoglyceride | | 1.2 |
| Fatty acid | | <1.0 |

### Example 8

### Comparison of the Products from Examples 6+7

| | | | Enrichment/ |
|---|---|---|---|
| | Starting material | Product | depletion |
| Example 6: | | | |
| Sum of omega-3 fatty acids | 37.6% | 50.2% | 1.33 |
| Sum of 5x- and 6x- | 33.4% | 45.4% | 1.36 |
| unsaturated fatty acids | | | |
| Sum of saturated fatty acids | 26.7% | 12.2% | 2.19 |
| (C14:0, C16:0 and C18:0) | | | |
| Example 7: | | | |
| Sum of omega-3 fatty acids | 37.6% | 48.1% | 1.28 |
| Sum of 5x- and 6x- | 33.4% | 43.5% | 1.3 |
| unsaturated fatty acids | | | |
| Sum of saturated fatty acids | 26.7% | 22.6% | 1.18 |
| (C14:0, C16:0 and C18:0) | | | |

It can clearly be seen that, for a comparable omega-3 PUFA enrichment and a comparable glyceride distribution, a distinctly reduced concentration of saturated fatty acids in the product is obtained where lipase A from Candida antarctica (Example 6) is used as opposed to lipase A from Candida cylindracea (Example 7). This can also be compared via the corresponding enrichment or depletion factor which was calculated as follows:

enrichment factor omega-3 fatty acids=product [%]/starting material [%]

depletion factor saturated fatty acids=starting material=[%]/product [%]

### Example 9

### Selective Hydrolysis of Fish Oil 18/12 with Lipase A from Candida Antarctica and from Candida Cylindracea

In a reactor, 1,000 g fish oil 18/12 (a fish oil with the composition shown in the following Table) and 500 g water were heated with stirring to 45° C. 25 g immobilized lipase from Candida A (as in Example 6) were added and the whole was incubated overnight. 1 g lipase from Candida cylindracea (Lipomod®) was then added and the whole was incubated until an acid value of >100 was reached. The crude product had an acid value of 112. The reaction mixture was worked up in the same way as in Examples 6 and 7. The results are set out in Table 10.

TABLE 10
fatty acid distribution and composition in the glyceride and in the
starting product

| Fatty acid | Fish oil 18/12 [%] | PUFA glyceride [%] |
|---|---|---|
| 14:0 | 6.7 | 2.6 |
| 16:0 | 16.6 | 7.8 |
| 16:1 | 8.0 | 4.0 |
| 16:4 | 2.1 | 2.4 |
| 18:0 | 3.4 | 2.1 |
| 18:1 | 12.2 | 8.1 |
| 18:2 | 1.3 | 1.0 |
| 18:3 | 0.9 | 0.6 |
| 18:4 | 3.3 | 4.3 |
| 20:1 | 1.5 | 1.3 |
| 20:4 | 1.1 | 1.7 |
| 20:5 | 18.4 | 26.5 |
| 22:5 | 2.2 | 3.2 |
| 22:6 | 12.8 | 24.3 |

### Example 10

### Selective Hydrolysis of Brudy Algatrium Tuna Oil with Lipase A from Candida Antarctica and from Candida Cylindracea

The reaction was carried out as in Example 9 with 1,000 g Brudy Algatrium DHA 18 tuna oil (a fish oil from tuna with the fatty acid composition specified below commercially obtainable under the name of Algatrium® DHA 18 from Proyecto Empresarial Brudy, Barcelona, Spain). The crude product had an acid value of 104. The results are set out in Table 11.

TABLE 11
fatty acid distribution and composition in the glyceride and in the
starting product

| | Fish oil | |
|---|---|---|
| Fatty acid | DHA 18 [%] | PUFA glyceride [%] |
| 14:0 | 4.1 | 1.6 |
| 16:0 | 18.3 | 5.4 |
| 16:1 | 5.3 | 2.7 |
| 18:0 | 4.9 | 2.1 |
| 18:1 | 17.8 | 10.2 |
| 18:2 | 1.8 | 1.1 |
| 18:3 | 0.7 | |
| 18:4 | 1.2 | 1.7 |
| 20:1 | 2.4 | 2.2 |
| 20:4 | 1.8 | 3.0 |
| 20:5 | 8.5 | 12.2 |
| 22:5 | 1.7 | 2.7 |
| 22:6 | 21.6 | 44.3 |

### Example 11

### Selective Hydrolysis of Brudy Algatrium Tuna Oil with Lipase from Candida Cylindracea

In a reactor, 800 g Brudy Algatrium DHA 18 tuna oil and 200 g water were heated with stirring to 45° C. 2 g lipase from Candida cylindracea (Lipomod® 34) were added and the whole was incubated until an acid value of >100 was reached. The crude product had an acid value of 122. The reaction mixture was worked up in the same way as in Examples 6 and 7. The results are set out in Table 12.

TABLE 12
fatty acid distribution and composition in the glyceride and in the
starting product

| Fatty acid | Fish oil DHA [%] | PUFA glyceride [%] |
|---|---|---|
| 14:0 | 4.1 | 2.3 |
| 16:0 | 18.3 | 10.4 |
| 16:1 | 5.3 | 3.0 |
| 18:0 | 4.9 | 3.0 |
| 18:1 | 17.8 | 10.4 |
| 18:2 | 1.8 | 1.0 |
| 18:3 | 0.7 | 0.4 |
| 18:4 | 1.2 | 1.2 |
| 20:1 | 2.4 | 1.9 |
| 20:4 | 1.8 | 2.4 |
| 20:5 | 8.5 | 7.4 |
| 22:5 | 1.7 | 2.6 |
| 22:6 | 21.6 | 45.2 |

### Example 12

### Selective Hydrolysis of Lamotte Type 170 Fish Oil with Lipase from Candida Cylindracea

The reaction was carried out as in Example 7 with 800 g Lamotte Type 170 fish oil (a fish oil with the fatty acid composition shown below obtainable under the name of Type 170 from Henry Lamotte GmbH, Bremen). The crude product had an acid value of 111. The results are set out in Table 13.

TABLE 13
fatty acid distribution and composition in the glyceride and in the
starting product

| | Type 170 | |
|---|---|---|
| Fatty acid | fish oil [%] | PUFA glyceride [%] |
| 14:0 | 5.0 | 2.5 |
| 16:0 | 18.2 | 9.4 |
| 16:1 | 6.1 | 3.0 |
| 18:0 | 4.6 | 2.8 |
| 18:1 | 16.8 | 9.2 |
| 18:2 | 1.5 | 0.8 |
| 18:3 | 0.7 | 0.4 |
| 18:4 | 1.4 | 1.5 |
| 20:1 | 2.4 | 2.3 |
| 20:4 | 1.6 | 2.1 |
| 20:5 | 10.5 | 9.9 |
| 22:5 | 1.9 | 3.1 |
| 22:6 | 19.3 | 38.8 |

### Example 13

### Comparison of the Products from Examples 9 to 12: Selectivity for Saturated Fatty Acids

| | | | Enrichment/ |
|---|---|---|---|
| | Starting material | Product | depletion |
| Example 9: | | | |
| Sum of omega-3 fatty acids | 37.6% | 58.9% | 1.57 |
| Sum of saturated fatty acids | 26.7% | 12.5% | 2.14 |
| (14, 16, 18) | | | |
| Sum of saturated fatty acids | | 40.6% | |
| in the distillate | | | |
| Example 10: | | | |
| Sum of omega-3 fatty acids | 33.7% | 61.4% | 1.82 |
| Sum of saturated fatty acids | 27.3% | 9.1% | 3.0 |
| (14, 16, 18) | | | |
| Sum of saturated fatty acids | | 44.5% | |
| in the distillate | | | |
| Example 11: | | | |
| Sum of omega-3 fatty acids | 33.7% | 57.2% | 1.7 |
| Sum of saturated fatty acids | 27.3% | 15.7% | 1.74 |
| (14, 16, 18) | | | |
| Sum of saturated fatty acids | | 31.3% | |
| in the distillate | | | |
| Example 12: | | | |
| Sum of omega-3 fatty acids | 33.8% | 54.1% | 1.6 |
| Sum of saturated fatty acids | 27.8% | 14.7% | 1.89 |
| (14, 16, 18) | | | |
| Sum of saturated fatty acids | | 35.1% | |
| in the distillate | | | |

The sum of C14:0, C16:0 and C18:0 was used as the saturated fatty acids.

It can clearly be seen that, for a comparable omega-3 PUFA enrichment, a distinctly reduced concentration of saturated fatty acids in the product is obtained where a mixture of lipase A from Candida antarctica and lipase from Candida cylindracea (Examples 9 and 10) is used as opposed to lipase from Candida cylindracea lipase alone (Examples 11 and 12). The difference in selectivity is reflected in particular in the percentage of fatty acids released (distillate). This can also be compared via the corresponding enrichment or depletion factor which was calculated as follows:

enrichment factor omega-3 fatty acids=product [%]/starting material [%]

depletion factor saturated fatty acids=starting material [%]/product [%]

### Example 14

### Comparison of the Products from Examples 9 to 12: Selectivity for PUFAs

| | Starting material | Product |
|---|---|---|
| Example 9: | | |
| Sum of 5x- and 6x-unsaturated fatty acids | 33.4% | 54% |
| Sum of eicosapentaenoic acid (EPA, C20:5) | 18.4% | 26.5% |
| Sum of docosahexaneoic acid (DHA, C22:6) | 12.8% | 24.3% |
| Example 10: | | |
| Sum of 5x- and 6x-unsaturated fatty acids | 31.8% | 59.2% |
| Sum of eicosapentaenoic acid (EPA, C20:5) | 8.5% | 12.2% |
| Sum of docosahexaneoic acid (DHA, C22:6) | 21.6% | 44.3% |
| Example 11: | | |
| Sum of 5x- and 6x-unsaturated fatty acids | 31.8% | 55.2% |
| Sum of eicosapentaenoic acid (EPA, C20:5) | 8.5% | 7.4% |
| Sum of docosahexaneoic acid (DHA, C22:6) | 21.6% | 45.2% |
| Example 12: | | |
| Sum of 5x- and 6x-unsaturated fatty acids | 31.7% | 51.8% |
| Sum of eicosapentaenoic acid (EPA, C20:5) | 10.5% | 9.9% |
| Sum of docosahexaneoic acid (DHA, C22:6) | 19.3% | 38.8% |

It can clearly be seen that, where a mixture of lipase A from Candida antarctica and lipase from Candida cylindracea was used, the omega-3 PUFA enrichment was greater than where Candida cylindracea lipase was used on its own. The difference in selectivity is attributable in particular to the higher selectivity of lipase A from Candida antarctica for EPA which becomes significant at degrees of hydrolysis in the oil of >50%.

### Example 15

### Selective Hydrolysis of Napro 18/12 Fish Oil with Candida Antarctica A and Candida Cylindracea Lipase

In a reactor, 3,500 g fish oil 18/12 (Napro Pharma) and 1,750 g water were heated with stirring to 45° C. 87.5 g immobilized Candida A lipase (as in Example 6) were then added and the whole was incubated overnight. 2.5 g Candida cylindracea lipase (Lipomod 34) were then added and the whole was incubated for 4 h. The crude product had an acid value of 96.

After phase separation, the aqueous phase was removed and the oil phase was dried at 80° C. in a rotary evaporator. The oil phase was worked up by short-path distillation. The free acids were distilled overhead at a temperature of 190° C. and a vacuum of 0.3 mbar. The distillation was carried out twice. The bottom product was the enriched PUFA glyceride. The samples were analyzed for their fatty acid distribution by gas chromatography and for their glyceride composition by GPC.

TABLE 14
PUFA enrichment on the basis of Napro 18/12 fish oil

| | | |
|---|---|---|
| | | PUFA-glyceride |
| | Fish oil 18/12 | depletion/enrichment |
| Fatty acid | [%] | [%] |

| | | | |
|---|---|---|---|
| 14:0 | 6.7 | 2.5 | |
| 16:0 | 16.6 | 5.9 | |
| 16:1 | 8.0 | 4.4 | |
| 16:4 | 2.1 | 3.5 | |
| 18:0 | 3.4 | 1.4 | |
| 18:1 | 12.2 | 7.7 | |
| 18:2 | 1.3 | 0.9 | |
| 18:3 | 0.9 | 0.5 | |
| 18:4 | 3.3 | 4.5 | |
| 20:1 | 1.5 | 0.9 | |
| 20:4 | 1.1 | 1.7 | |
| 20:5 | 18.4 | 28.9 | |
| 22:5 | 2.2 | 3.5 | |
| 22:6 | 12.8 | 22.9 | |
| Sum of SAFA | 26.7 | 9.8 | 2.7 |
| (14, 16, 18) | | | |
| Sum of MUFA | 21.7 | 13 | 1.7 |
| Sum of PUFA | 40.8 | 65.5 | 1.6 |
| Sum of omega-3 | 37.6 | 60.3 | 1.6 |
| Sum of 5x- and | 33.4 | 55.3 | 1.7 |
| 6x-UFA | | | |
| Assigned | 90.5 | 89.2 | |
| Triglyceride | >95 | 66.5 | |
| Diglyceride | | 31.1 | |
| Monoglyceride | | 2.2 | |
| Fatty acid | | 0.3 | |

SAFA = saturated fatty acids
MUFA = monounsaturated fatty acids
UFA = unsaturated fatty acids

The enrichment factors were determined as in Example 13.

### Example 16

### Alkaline Hydrolysis of the Enriched Glycerides from Example 16

1,250 g dried glyceride fraction were introduced with 500 g ethanol into a reactor equipped with a stirrer, dropping funnel and reflux condenser and heated with stirring until the ethanol refluxed. 1,250 g of a potassium hydroxide solution consisting of 937.5 g water and 312.5 g KOH were then added dropwise over a period of 1 hour and the mixture wax refluxed for another 3 hours. Another 875 g water were introduced into the reactor, after which 575 g concentrated phosphoric acid were added over a period of 30 mins. For neutralization, the mixture was stirred for another 30 mins., after which the stirrer was switched off. After phase separation, the aqueous phase was removed through a bottom outlet valve. The oil phase was washed twice with 2,500 g water and then dried in a rotary evaporator.

Result: The neutralized and dried fatty acids had an acid value of 192. The fatty acid spectrum corresponded to that of the enriched glycerides used.

### Example 17

### Alkaline Transesterification of the Enriched Glycerides from Example 16 and PUFA Enrichment of the Ethyl Ester Fraction by Distillation

500 g dried glyceride fraction were introduced with 200 g ethanol into a reactor equipped with a stirrer, dropping funnel and reflux condenser and heated with stirring under nitrogen to a temperature of 60° C. 20 g 21% by weight sodium ethylate in ethanol were added and the reaction was maintained for 6 h at 60° C. After 2 h, another 10 g sodium ethylate solution were added. After 6 hours, 2% citric acid was added to the reaction mixture until the pH of the reaction mixture was below pH 6. The stirrer was switched off and, after phase separation, the oil phase was removed from the aqueous phase and dried in a rotary evaporator. The oil phase was worked by short-path distillation. Part of the relatively short-chain fatty acids (chain length </=C18) was distilled off at a temperature of 145° C. and a vacuum of 0.3 mbar. The bottom product was then completely distilled off overhead at a temperature of 185° C. and a vacuum of 0.3 mbar. The distillate was analyzed for its fatty acid distribution by gas chromatography.

TABLE 15
PUFA enrichment on the basis of Napro 18/12 fish oil

| | PUFA | Ethyl ester [%] |
|---|---|---|
| | glyceride [%] | enriched |
| Fatty acid | from Example 16 | by distillation |
| 14:0 | 2.5 | 0.4 |
| 16:0 | 5.9 | 2.8 |
| 16:1 | 4.4 | 1.9 |
| 17:0 | 3.5 | 1.4 |
| 18:0 | 1.4 | 1.3 |
| 18:1 | 7.7 | 7.0 |
| 18:2 | 0.9 | 0.8 |
| 18:3 | 0.5 | 0.6 |
| 18:4 | 4.5 | 3.8 |
| 20:1 | 0.9 | 1.2 |
| 20:4 | 1.7 | 2.0 |
| 20:5 | 28.9 | 33.8 |
| 22:5 | 3.5 | 4.8 |
| 22:6 | 22.9 | 29.7 |
| Sum of SAFA | 13.3 | 5.9 |
| Sum of MUFA | 13.1 | 10.1 |
| Sum of PUFA | 63.1 | 75.5 |
| Sum of omega-3 | 60.4 | 72.7 |
| Assigned | 90.4 | 92.7 |

### Example 18

### Comparative Hydrolysis of Napro 18/12 Fish Oil

Quantities of 50 g water and 50 g Napro 18/12 fish oil were introduced into 5 flasks equipped with stirring “fishes”. 120 U Candida antarctica A lipase were added to mixture 1. 120 U Candida rugosa lipase were added to mixture 2. A mixture of Candida antarctica A lipase and Candida rugosa lipase with a total activity of 120 U was added to mixture 3. 120 U Candida cylindracea lipase were added to mixture 4. A mixture of Candida antarctica A lipase and Candida cylindracea lipase with a total activity of 120 U was added to mixture 5. The mixtures were incubated with stirring at 45° C. Samples were taken from the mixtures after 1 h, 2 h, 3 h, 5 h, 7 h, 24 h and 48 h. The oil phase was separated from the water phase and the samples were analyzed for their acid value. The corresponding degree of hydrolysis of the fish oils was calculated from the measured acid value on the basis of a maximum attainable acid value of 195 in the event of complete hydrolysis.

TABLE 16
degree of hydrolysis [in %] as a function of reaction time

| Time | Mixture 1 | Mixture 2 | Mixture 3 | Mixture 4 | Mixture 5 |
|---|---|---|---|---|---|
| 0 h | 0% | 0% | 0% | 0% | 0% |
| 1 h | 14.5% | 17.9% | 20.2% | 28.1% | 23% |
| 2 h | 19% | 23% | 23.4% | 32.8% | 28.5% |
| 3 h | 23.5% | 26% | 27.6% | 37% | 33.3% |
| 5 h | 26.7% | 28.3% | 30.8% | 38.9% | 37.4% |
| 7 h | 30.2% | 30.7% | 35.2% | 42.2% | 42.9% |
| 24 h | 39.8% | 38.7% | 42.7% | 48.3% | 51.1% |
| 48 h | 42.6% | 40.4% | 46.4% | 49.8% | 54% |

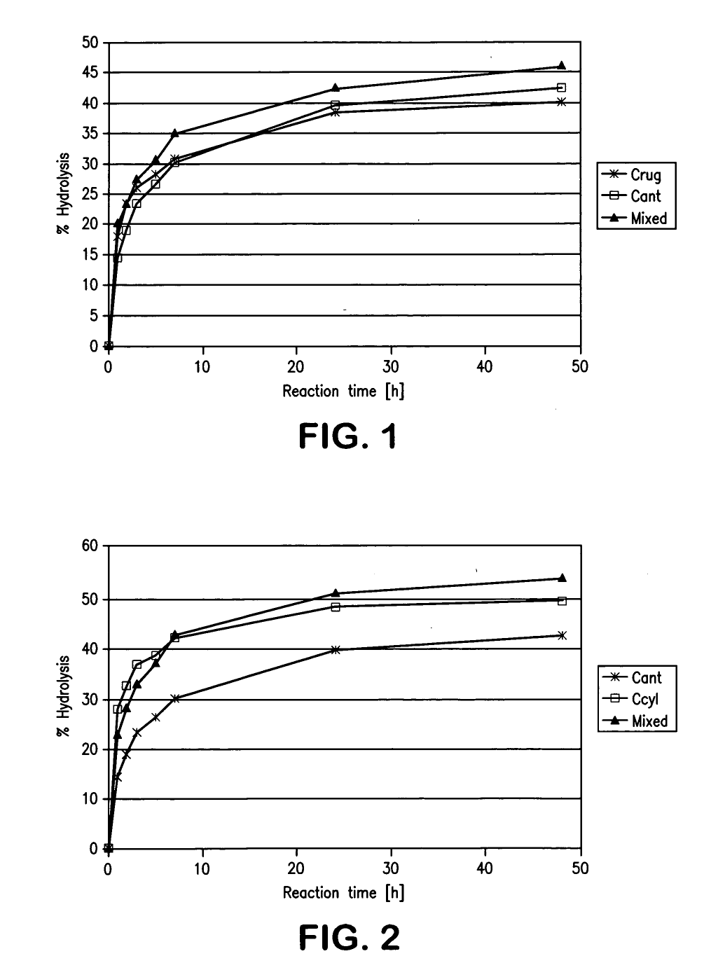

FIG. 1: The degrees of hydrolysis of mixtures 1 and 2 were graphically compared with mixture 3 which contains the enzyme mixture for the same overall enzyme activity (Crug=Candida rugosa lipase (mixture 2), Cant=Candida antarctica A lipase (mixture 1) and Mixed=mixture of both lipases (mixture 3)).
FIG. 2: The degrees of hydrolysis of mixtures 1 and 4 were graphically compared with mixture 5 which contains the enzyme mixture for the same overall enzyme activity (Ccyl=Candida cylindracea lipase (mixture 4), Cant=Candida antarctica A lipase (mixture 1) and Mixed=mixture of both lipases (mixture 5)).
Result: Both the mixture of Candida antarctica A lipase with Candida rugosa lipase and the mixture of Candida antarctica A lipase with Candida cylindracea lipase showed a higher hydrolysis rate than the respective enzymes on their own for the same overall enzyme activity in the reaction mixture. The synergistic effect of the mixture of Candida antarctica A and Candida rugosa was evident over the entire hydrolysis period whereas the mixture containing Candida cylindracea only produced a synergistic effect of significance from a degree of hydrolysis of 40-50%.

### Example 19

### Comparative Hydrolysis of Lamotte Type 170 Fish Oil

Quantities of 50 g water and 50 g Lamotte Type 170 fish oil were introduced into 5 flasks equipped with stirring “fishes”. 120 U Candida antarctica A lipase were added to mixture 1. 120 U Candida rugosa lipase were added to mixture 2. A mixture of Candida antarctica A lipase and Candida rugosa lipase with a total activity of 120 U was added to mixture 3. 120 U Candida cylindracea lipase were added to mixture 4. A mixture of Candida antarctica A lipase and Candida cylindracea lipase with a total activity of 120 U was added to mixture 5. The mixtures were incubated with stirring at 45° C. Samples were taken from the mixtures after 1 h, 2 h, 3 h, 5 h, 7 h, 24 h and 48 h. The oil phase was separated from the water phase and the samples were analyzed for their acid value. The corresponding degree of hydrolysis of the fish oils was calculated from the measured acid value on the basis of a maximum attainable acid value of 195 in the event of complete hydrolysis.

TABLE 17
degree of hydrolysis [in %] as a function of reaction time

| Time | Mixture 1 | Mixture 2 | Mixture 3 | Mixture 4 | Mixture 5 |
|---|---|---|---|---|---|
| 0 h | 0% | 0% | 0% | 0% | 0% |
| 1 h | 17.3% | 13.5% | 18% | 25.4% | 16.2% |
| 2 h | 22.9% | 17.8% | 25.3% | 31% | 22.3% |
| 3 h | 26% | 23.5% | 27.6% | 37% | 33.3% |
| 5 h | 28% | 26.9% | 33.5% | 37.6% | 36.5% |
| 7 h | 32.9% | 32% | 39.6% | 39.6% | 41.6% |
| 24 h | 35.6% | 36.4% | 41.5% | 43.1% | 43.6% |
| 48 h | 38.5% | 39.8% | 42.7% | 45.1% | 46.2% |

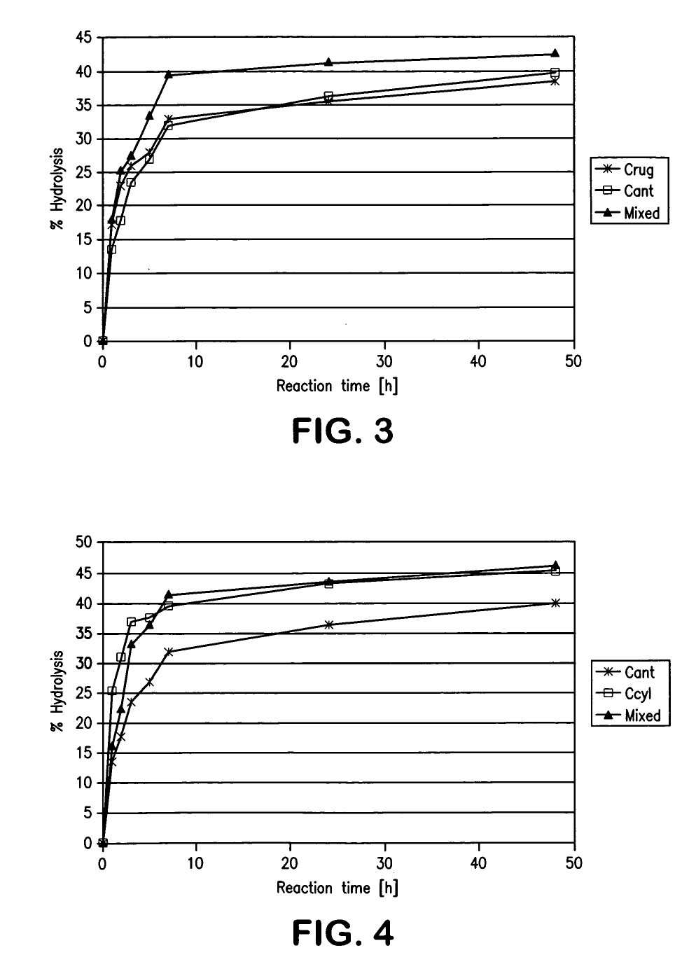

FIG. 3: The degrees of hydrolysis of mixtures 1 and 2 were graphically compared with mixture 3 which contains the enzyme mixture for the same overall enzyme activity (Crug=Candida rugosa lipase (mixture 2), Cant=Candida antarctica A lipase (mixture 1) and Mixed=mixture of both lipases (mixture 3)).
FIG. 4: The degrees of hydrolysis of mixtures 1 and 4 were graphically compared with mixture 5 which contains the enzyme mixture for the same overall enzyme activity (Ccyl=Candida cylindracea lipase (mixture 4), Cant=Candida antarctica A lipase (mixture 1) and Mixed=mixture of both lipases (mixture 5)).
Result: Both the mixture of Candida antarctica A lipase with Candida rugosa lipase and the mixture of Candida antarctica A lipase with Candida cylindracea lipase showed a higher hydrolysis rate than the respective enzymes on their own for the same overall enzyme activity in the reaction mixture. The synergistic effect of the mixture of Candida antarctica A and Candida rugosa was evident over the entire hydrolysis period whereas the mixture containing Candida cylindracea only produced a synergistic effect of significance from a degree of hydrolysis of 40-50%.

### Example 20

### Comparative Hydrolysis of Napro 18/12 Fish Oil

Mixtures 1, 2 and 3 from Example 18 were compared for their fatty acid composition. To this end, the samples taken from the reaction after 1 h, 2 h, 3 h, 5 h, 7 h, 24 h and 48 h were silylated and analyzed by gas chromatography. From the spectrum of the fatty acids released combined with the degree of hydrolysis determined on the basis of acid value, the composition of the fatty acids released and—by calculation—the composition of the fatty acid bound to the glyceride were determined. The starting composition of the Napro 18/12 fish oil was used as the basis for the calculation. The totaled fatty acid distributions of omega-3 fatty acids (C18:3, C18:4, C20:5, C22:5 and C22:6), saturated fatty acids and the sum of the saturated and unsaturated fatty acids with a chain length of C14 to C18 are shown in the following.

TABLE 18
plotting the fatty acid compositions in the glyceride fraction and
in the fatty acids released against the degree of hydrolysis (in %)

| Degree of | Omega-3 fatty | Omega-3 | Saturated | Saturated | C14-C18 | C14-C18 |
|---|---|---|---|---|---|---|
| hydrolysis | acid | glyceride | fatty acid | glyceride | fatty acid | glyceride |
| Mixture 1 | | | | | | |
| 0 | | 37.6 | | 29.3 | | 55 |
| 14.5 | 2 | 43.7 | 82.9 | 20.2 | 95.9 | 48 |
| 19 | 5.1 | 45.2 | 82.1 | 16.9 | 93 | 46.1 |
| 23.5 | 4.8 | 47.7 | 74 | 15.6 | 92.1 | 43.6 |
| 26.7 | 6.6 | 48.9 | 68.4 | 15.1 | 88.5 | 42.8 |
| 30.2 | 6.8 | 50.9 | 58.5 | 16.7 | 88.8 | 40.4 |
| 39.8 | 11.1 | 55.1 | 55.1 | 12.3 | 84.6 | 35.5 |
| 42.6 | 11.2 | 57.2 | 51.1 | 13.1 | 82 | 35 |
| Mixture 2 | | | | | | |
| 0 | | 37.6 | | 29.3 | | 55 |
| 17.9 | 4.1 | 44.9 | 35.5 | 27.9 | 94.9 | 46.3 |
| 23 | 3.5 | 47.8 | 40.8 | 25.9 | 95.9 | 42.8 |
| 26 | 5.2 | 49 | 40.9 | 25.2 | 94.2 | 41.2 |
| 28.3 | 5 | 50.4 | 43.1 | 23.6 | 93.5 | 39.8 |
| 30.7 | 6.2 | 51.5 | 42.5 | 23.4 | 92.6 | 38.4 |
| 38.7 | 7.9 | 56.3 | 44.5 | 19.7 | 89.8 | 33.1 |
| 40.4 | 9.6 | 56.6 | 44.2 | 19.2 | 89.1 | 31.9 |
| Mixture 3 | | | | | | |
| 0 | | 37.6 | | 29.3 | | 55 |
| 20.2 | 3.1 | 46.3 | 61.4 | 21.2 | 96.3 | 44.5 |
| 23.4 | 3.9 | 47.9 | 61.3 | 19.5 | 94.6 | 42.9 |
| 27.6 | 4.1 | 50.3 | 58.1 | 18.3 | 93.8 | 40.3 |
| 30.8 | 5.3 | 52 | 56.7 | 17.1 | 92.1 | 38.5 |
| 35.2 | 7.1 | 54.1 | 49 | 18.6 | 89.2 | 36.4 |
| 42.7 | 9.9 | 58.3 | 48.5 | 15 | 87.4 | 30.8 |
| 46.4 | 10.4 | 61.1 | 46.3 | 14.6 | 84.5 | 29.4 |

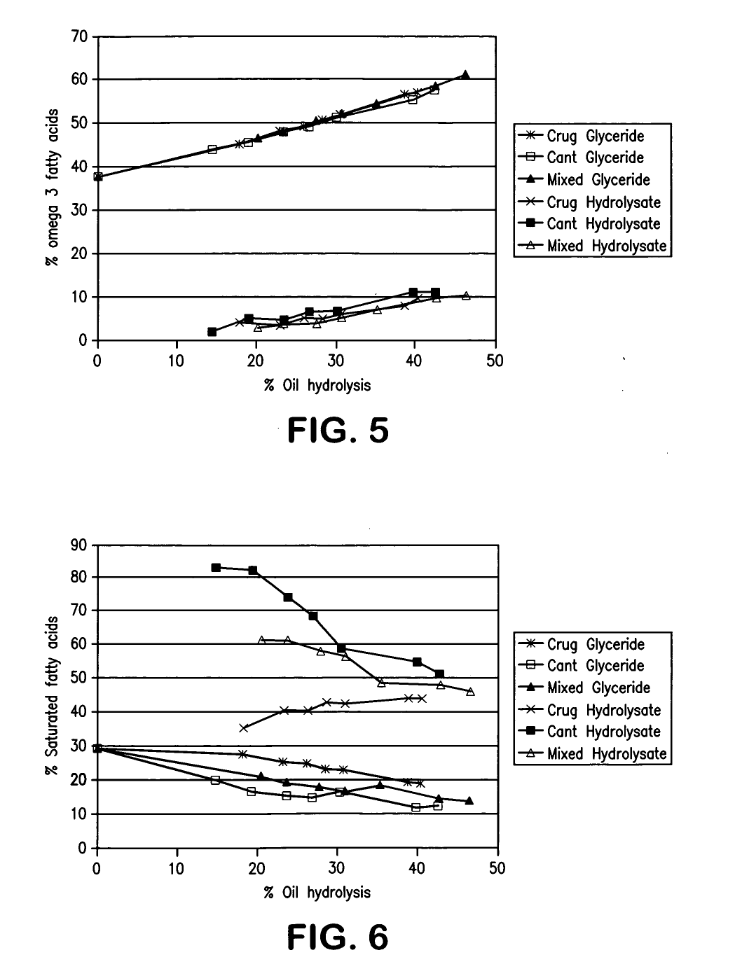

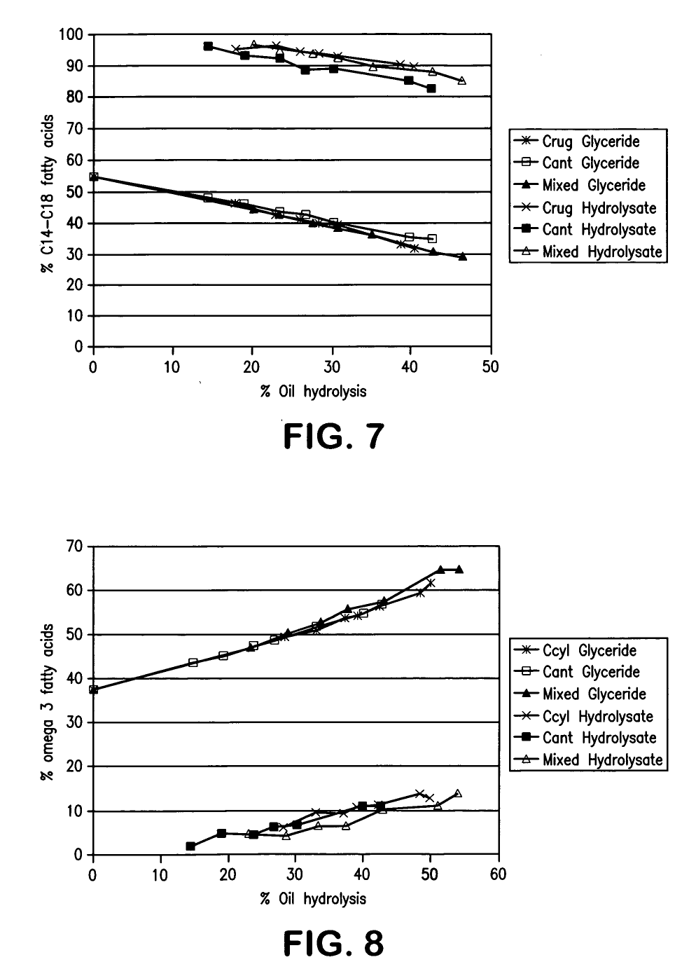

FIG. 5: Comparative plotting of the omega-3 fatty acids of the individual mixtures in the glyceride and fatty acid fraction (Cant hydrolyzate=fatty acids released from mixture 1; Cant glyceride=glyceride-bound fatty acids from mixture 1, Crug hydrolyzate=fatty acids released from mixture 2; Crug glyceride=glyceride-bound fatty acids from mixture 2, mixed hydrolyzate=fatty acids released from mixture 3; mixed glyceride=glyceride-bound fatty acids from mixture 3).
FIG. 6: Comparative plotting of the saturated fatty acids of the individual mixtures in the glyceride and fatty acid fraction (Cant hydrolyzate=fatty acids released from mixture 1; Cant glyceride=glyceride-bound fatty acids from mixture 1, Crug hydrolyzate=fatty acids released from mixture 2; Crug glyceride=glyceride-bound fatty acids from mixture 2, mixed hydrolyzate=fatty acids released from mixture 3; mixed glyceride=glyceride-bound fatty acids from mixture 3).
FIG. 7: Comparative plotting of the saturated and unsaturated C14-C18 fatty acids of the individual mixtures in the glyceride and fatty acid fraction (Cant hydrolyzate=fatty acids released from mixture 1; Cant glyceride=glyceride-bound fatty acids from mixture 1, Crug hydrolyzate=fatty acids released from mixture 2; Crug glyceride=glyceride-bound fatty acids from mixture 2, mixed hydrolyzate=fatty acids released from mixture 3; mixed glyceride=glyceride-bound fatty acids from mixture 3).
Result: The release of C14-C18 fatty acids from the glyceride in total was comparable for both the enzymes used, as was the negative selectivity for omega-3 fatty acids mainly containing eicosapentaenoic acid and docosahexaenoic acid. The mixture of the two enzymes showed comparable behavior to the individual enzymes. A clear difference was visible in the release of the saturated fatty acids from the glyceride. A distinct preference on the part of Candida antarctica A lipase was discernible here. Candida rugosa lipase showed a corresponding preference for the unsaturated C14-C18 fatty acids which explains the similar trend of the C14-C18 fatty acids as a whole. This difference produced the synergistic effect of the two enzymes in relation to the increased hydrolysis rate as shown in Example 5.

### Example 21

### Comparative Hydrolysis of Napro 18/12 Fish Oil

Mixtures 1, 4 and 5 from Example 18 were compared for their fatty acid composition. To this end, the samples taken from the reaction after 1 h, 2 h, 3 h, 5 h, 7 h, 24 h and 48 h were silylated and analyzed by gas chromatography. From the spectrum of the fatty acids released combined with the degree of hydrolysis determined on the basis of acid value, the composition of the fatty acids released and—by calculation—the composition of the fatty acid bound to the glyceride were determined. The starting composition of the Napro 18/12 fish oil was used as the basis for the calculation. The totaled fatty acid distributions of omega-3 fatty acids (C18:3, C18:4, C20:5, C22:5 and C22:6), saturated fatty acids and the sum of the saturated and unsaturated fatty acids with a chain length of C14 to C18 are shown in the following.

TABLE 19
plotting the fatty acid compositions in the glyceride fraction and
in the fatty acids released against the degree of hydrolysis (in %)

| Degree of | Omega-3 fatty | Omega-3 | Saturated | Saturated | C14-C18 | C14-C18 |
|---|---|---|---|---|---|---|
| hydrolysis | acid | glyceride | fatty acid | glyceride | fatty acid | glyceride |
| Mixture 1 | | | | | | |
| 0 | | 37.6 | | 29.3 | | 55 |
| 14.5 | 2 | 43.7 | 82.9 | 20.2 | 95.9 | 48 |
| 19 | 5.1 | 45.2 | 82.1 | 16.9 | 93 | 46.1 |
| 23.5 | 4.8 | 47.7 | 74 | 15.6 | 92.1 | 43.6 |
| 26.7 | 6.6 | 48.9 | 68.4 | 15.1 | 88.5 | 42.8 |
| 30.2 | 6.8 | 50.9 | 58.5 | 16.7 | 88.8 | 40.4 |
| 39.8 | 11.1 | 55.1 | 55.1 | 12.3 | 84.6 | 35.5 |
| 42.6 | 11.2 | 57.2 | 51.1 | 13.1 | 82 | 35 |
| Mixture 4 | | | | | | |
| 0 | | 37.6 | | 29.3 | | 55 |
| 28.1 | 6.4 | 49.8 | 39.8 | 25.2 | 92.6 | 40.3 |
| 32.8 | 9.7 | 51.2 | 37.8 | 25.1 | 87.3 | 39.2 |
| 37 | 9.6 | 54 | 39.2 | 23.5 | 86.3 | 36.7 |
| 38.9 | 11.2 | 54.5 | 38.2 | 23.6 | 84.7 | 36 |
| 42.2 | 116 | 56.6 | 35.6 | 24.7 | 85.2 | 33 |
| 48.3 | 14.1 | 59.6 | 39.3 | 20 | 81.5 | 30.3 |
| 49.8 | 13.2 | 61.9 | 40.4 | 18.2 | 82.9 | 27.3 |
| Mixture 5 | | | | | | |
| 0 | | 37.6 | | 29.3 | | 55 |
| 23 | 5 | 47.3 | 55.4 | 21.5 | 92.9 | 43.7 |
| 28.5 | 4.6 | 50.7 | 55 | 19.1 | 94.1 | 39.5 |
| 33.3 | 6.8 | 53 | 50.9 | 18.5 | 90 | 37.5 |
| 37.4 | 6.9 | 56 | 51.1 | 16.2 | 90.3 | 33.9 |
| 42.9 | 10.7 | 57.8 | 46 | 16.7 | 85 | 32.5 |
| 51.1 | 11.6 | 64.9 | 44.5 | 13.4 | 84.2 | 24.5 |
| 54 | 14.3 | 65 | 40.9 | 15.7 | 80.3 | 25.2 |

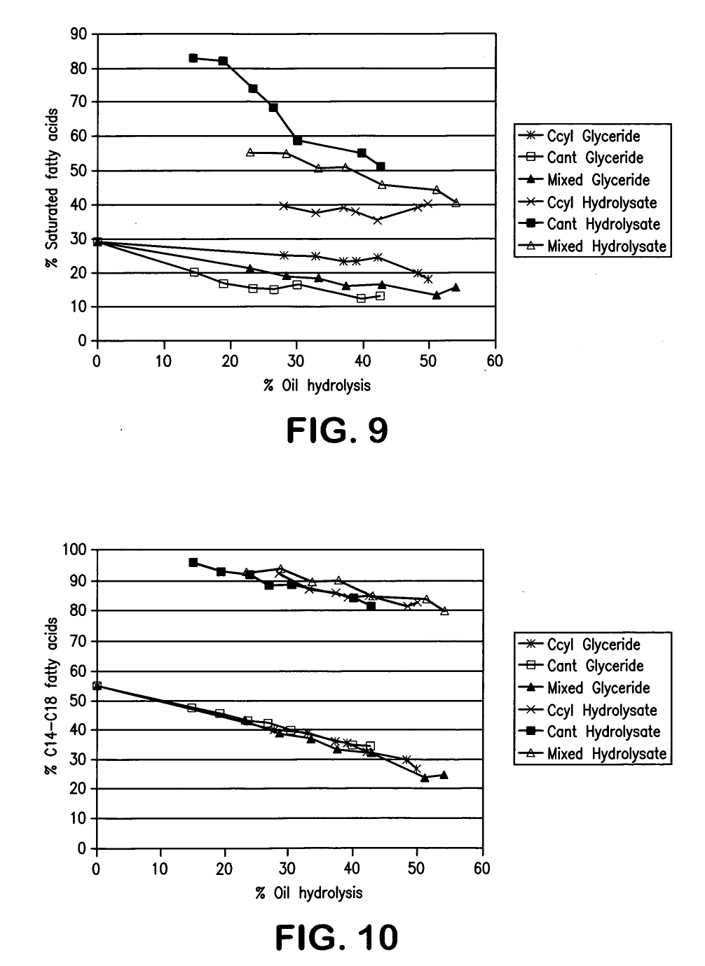

FIG. 8: Comparative plotting of the omega-3 fatty acids of the individual mixtures in the glyceride and fatty acid fraction (Cant hydrolyzate=fatty acids released from mixture 1; Cant glyceride=glyceride-bound fatty acids from mixture 1, Ccyl hydrolyzate=fatty acids released from mixture 4; Ccyl glyceride=glyceride-bound fatty acids from mixture 4, mixed hydrolyzate=fatty acids released from mixture 5; mixed glyceride=glyceride-bound fatty acids from mixture 5).
FIG. 9: Comparative plotting of the saturated fatty acids of the individual mixtures in the glyceride and fatty acid fraction (Cant hydrolyzate=fatty acids released from mixture 1; Cant glyceride=glyceride-bound fatty acids from mixture 1, Ccyl hydrolyzate=fatty acids released from mixture 4; Ccyl glyceride=glyceride-bound fatty acids from mixture 4, mixed hydrolyzate=fatty acids released from mixture 5; mixed glyceride=glyceride-bound fatty acids from mixture 5).
FIG. 10: Comparative plotting of the saturated and unsaturated C14-C18 fatty acids of the individual mixtures in the glyceride and fatty acid fraction (Cant hydrolyzate=fatty acids released from mixture 1; Cant glyceride=glyceride-bound fatty acids from mixture 1, Ccyl hydrolyzate=fatty acids released from mixture 4; Ccyl glyceride=glyceride-bound fatty acids from mixture 4, mixed hydrolyzate=fatty acids released from mixture 5; mixed glyceride=glyceride-bound fatty acids from mixture 5).
Result: The release of C14-C18 fatty acids from the glyceride in total was comparable for both the enzymes used, as was the negative selectivity for omega-3 fatty acids mainly containing eicosapentaenoic acid and docosahexaenoic acid. The mixture of the two enzymes showed comparable behavior to the individual enzymes. A clear difference was visible in the release of the saturated fatty acids from the glyceride. A distinct preference on the part of Candida antarctica A lipase was discernible here. Candida rugosa lipase showed a corresponding preference for the unsaturated C14-C18 fatty acids which explains the similar trend of the C14-C18 fatty acids as a whole. This difference produced the synergistic effect of the two enzymes in relation to the increased hydrolysis rate as shown in Example 18.

### Example 22

### Comparative Hydrolysis of Lamotte Type 170 Fish Oil

Mixtures 1, 2, 3, 4 and 5 from Example 19 were compared for their fatty acid composition. To this end, the samples taken from the reaction after 1 h, 2 h, 3 h, 5 h, 7 h, 24 h and 48 h were silylated and analyzed by gas chromatography. From the spectrum of the fatty acids released combined with the degree of hydrolysis determined on the basis of acid value, the composition of the fatty acids released and—by calculation—the composition of the fatty acid bound to the glyceride were determined. The starting composition of the Napro 18/12 fish oil was used as the basis for the calculation. The totaled fatty acid distributions of omega-3 fatty acids (C18:3, C18:4, C20:5, C22:5 and C22:6), saturated fatty acids and the sum of the saturated and unsaturated fatty acids with a chain length of C14 to C18 are shown in the following.

Result: The release of C14-C18 fatty acids from the glyceride in total was comparable for both the enzymes used, as was the negative selectivity for omega-3 fatty acids mainly containing eicosapentaenoic acid and docosahexaenoic acid. The mixture of the two enzymes showed comparable behavior to the individual enzymes. A clear difference was visible in the release of the saturated fatty acids from the glyceride. A distinct preference on the part of Candida antarctica A lipase was discernible here. Candida rugosa lipase and Candida cylindracea lipase showed a corresponding preference for the unsaturated C14-C18 fatty acids which explains the similar trend of the C14-C18 fatty acids as a whole. This difference produced the synergistic effect of the two enzymes in relation to the increased hydrolysis rate as shown in Example 19.

### Example 23

### Selective Transesterification of Napro 11/19 Fish Oil with Candida Antarctica A

### A) Enzyme Immobilization

80 g Lewatit VPOC 1600 (Lanxess) and 1500 ml water were introduced into a 2.5 liter reactor. 120 g of a commercially obtainable lipase A from Candida antarctica (Novozym® 735) were then added. The mixture was incubated for 3 h and then filtered off. The immobilizate was washed with water and used moist for the transesterification.

### B) Reaction+Working Up

1,000 g fish oil 11/19 (a fish oil with the composition shown in the following Table), 100 g water and 100 g ethanol were introduced into a double-jacketed reactor and heated with stirring to 45° C. The moist enzyme immobilizate was added to the mixture which was then incubated with stirring at a constant temperature for 24 h. The acid value in the product mixture was 15.6. After phase separation, the aqueous phase was removed and the oil phase was dried at 80° C. in a rotary evaporator. The oil phase was worked up by short-path distillation. Ethyl esters formed and free acids were distilled overhead at a temperature of 190° C. and a vacuum of 0.2 mbar. The sump product is the enriched PUFA glyceride. The mass balance of distillate and bottom product produced a degree of conversion of the oil of 50%, just under 20% being present as free acid and over 80% of the fatty acids released being present in the form of the ethyl esters.

### C) Product Analysis

The product and, for comparison, the starting material were analyzed by GPC (gel permeation chromatography) (glycide distribution); GC (gas chromatography) (fatty acid sample after methylation) and the acid value was measured. The results are set out in Table 20.

TABLE 20
fatty acid distribution and composition in the glyceride and in the
starting product

| | | PUFA-glyceride | |
|---|---|---|---|
| | Fish oil 11/19 | depletion/enrichment | |
| Fatty acid | [%] | [%] | |
| 14:0 | 5.0 | 1.3 | |
| 16:0 | 15.3 | 3.8 | |
| 16:1 | 5.8 | 5.1 | |
| 16:4 | 1.0 | 1.4 | |
| 18:0 | 3.6 | 1.4 | |
| 18:1 | 8.3 | 7.4 | |
| 18:4 | 3.9 | 6.2 | |
| 20:4 | 0.9 | 1.4 | |
| 20:5 | 14.0 | 22.5 | |
| 22:1 | 2.2 | 1.7 | |
| 22:5 | 1.8 | 3 | |
| 22:6 | 19.6 | 33.7 | |
| Sum of SAFA (14.16.18) | 23.9 | 6.5 | 3.7 |
| Sum of MUFA | 16.3 | 14.2 | 1.1 |
| Sum of PUFA | 41.2 | 68.2 | 1.7 |
| Sum of omega-3 | 39.3 | 65.4 | 1.7 |
| Sum of 5x- and 6x- | 35.4 | 59.2 | 1.7 |
| Unsaturated fatty acids | 81.4 | 88.9 | |
| Assigned | | | |
| Fatty acid + ethyl ester | | 1.5 | |
| Monoglyceride | | 6.1 | |
| Diglyceride | | 43.5 | |
| Triglyceride | | 48 | |
| Acid value | | 2.1 | |

### Example 24

### Selective Transesterification of Napro 11/19 Fish Oil with Candida Antarctica A

A reaction was carried out as in Example 23, followed by incubation for 48 h. The acid value in the product mixture was 27. The mass balance of distillate and bottom product produced a degree of conversion of the oil of 65%, about 20% being present as free acids and about 80% of the fatty acids released being present in the form of the ethyl esters. The results are set out in Table 16. The distillation was carried out under a vacuum of 0.06 mbar and at a temperature of 180° C.

TABLE 21
fatty acid distribution and composition in the glyceride and in the
starting product

| | | PUFA-glyceride | |
|---|---|---|---|
| | Fish oil 11/19 | depletion/enrichment | |
| Fatty acid | [%] | [%] | |
| 14:0 | 5.0 | 1.6 | |
| 16:0 | 15.3 | 4.7 | |
| 16:1 | 5.8 | 2.8 | |
| 16:4 | 1.0 | 1.6 | |
| 18:0 | 3.6 | 1.2 | |
| 18:1 | 8.3 | 4.9 | |
| 18:4 | 3.9 | 6.3 | |
| 20:4 | 0.9 | 1.4 | |
| 20:5 | 14.0 | 22.2 | |
| 22:5 | 1.8 | 2.9 | |
| 22:6 | 19.6 | 39.3 | |
| Sum of SAFA (14.16.18) | 23.9 | 7.5 | 3.2 |
| Sum of MUFA | 14.1 | 7.7 | 3.2 |
| Sum of PUFA | 41.2 | 73.7 | 1.8 |
| Sum of omega-3 | 39.3 | 70.7 | 1.8 |
| Sum of 5x- and 6x- | 35.4 | 64.4 | 1.8 |
| Unsaturated fatty acids | 79.2 | 88.9 | |
| Assigned | | | |
| Fatty acid + ethyl ester | | 0.8 | |
| Monoglyceride | | 2.6 | |
| Diglyceride | | 44.9 | |
| Triglyceride | | 51.7 | |

### Example 25

### Selective Transesterification of Napro 18/12 Fish Oil with Candida Antarctica A

A reaction was carried out with Napro 18/12 fish oil as in Example 23. The acid value in the product mixture was 24. The mass balance of distillate and bottom product produced a degree of conversion of the oil of 60%, about 20% being present as free acids and about 80% of the fatty acids released being present in the form of the ethyl esters. The results are set out in Table 17. The distillation was carried out under a vacuum of 0.06 mbar and at a temperature of 180° C.

TABLE 22
fatty acid distribution and composition in the glyceride and in the
starting product

| | | PUFA-glyceride | |
|---|---|---|---|
| | Fish oil 11/19 | depletion/enrichment | |
| Fatty acid | [%] | [%] | |
| Fatty acid | Napro 18/12 | Sample ex 18/12 | |
| 14:0 | 6.7 | 1.6 | |
| 16:0 | 16.6 | 4.2 | |
| 16:1 | 8 | 4.5 | |
| 16:4 | 2.1 | 3.1 | |
| 18:0 | 3.4 | 0.9 | |
| 18:1 | 12.2 | 6.5 | |
| 18:2 | 1.3 | 0.8 | |
| 18.3 | 0.9 | 0.6 | |
| 18.4 | 3.3 | 5.5 | |
| 20:1 | 1.5 | 0.7 | |
| 20:4 | 1.1 | 1.5 | |
| 20.5 | 18.4 | 30 | |
| 22:5 | 2.2 | 3.4 | |
| 22:6 | 12.8 | 27.5 | |
| Sum of SAFA (14.16.18) | 26.7 | 6.7 | 4 |
| Sum of MUFA | 21.7 | 11.7 | 1.9 |
| Sum of PUFA | 40.8 | 71.6 | 1.8 |
| Sum of omega-3 | 37.6 | 67 | 1.8 |
| Sum of 5x- and 6x- | 33.4 | 60.9 | 1.8 |
| Unsaturated fatty acids | 90.5 | 90.8 | |
| Assigned | | | |
| Fatty acid + ethyl ester | | 0.7 | |
| Monoglyceride | | 1.9 | |
| Diglyceride | | 45.4 | |
| Triglyceride | | 52.1 | |

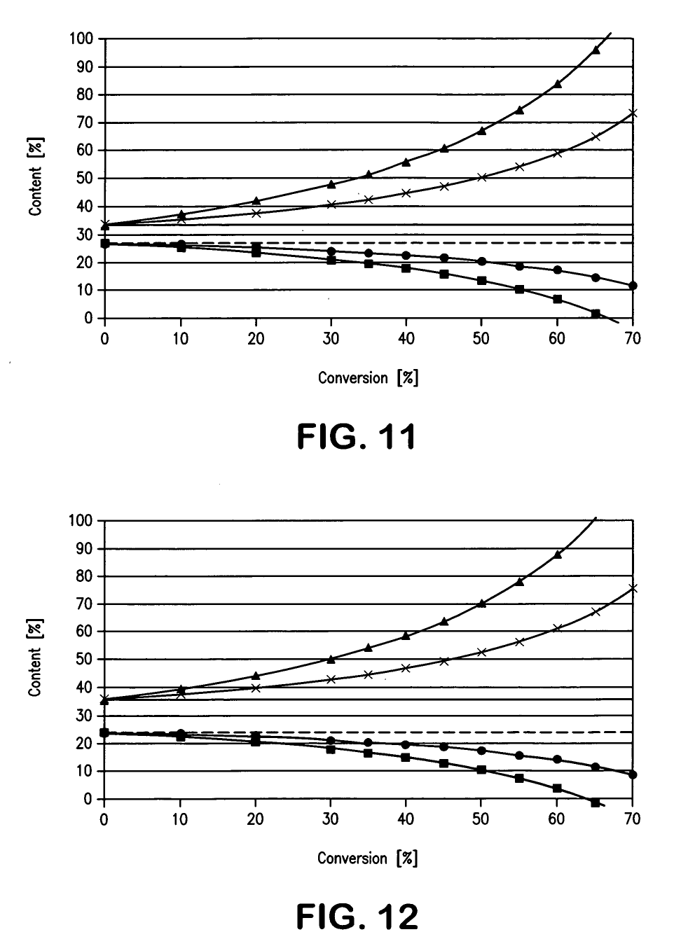

FIGS. 11 to 14 are described in the following:

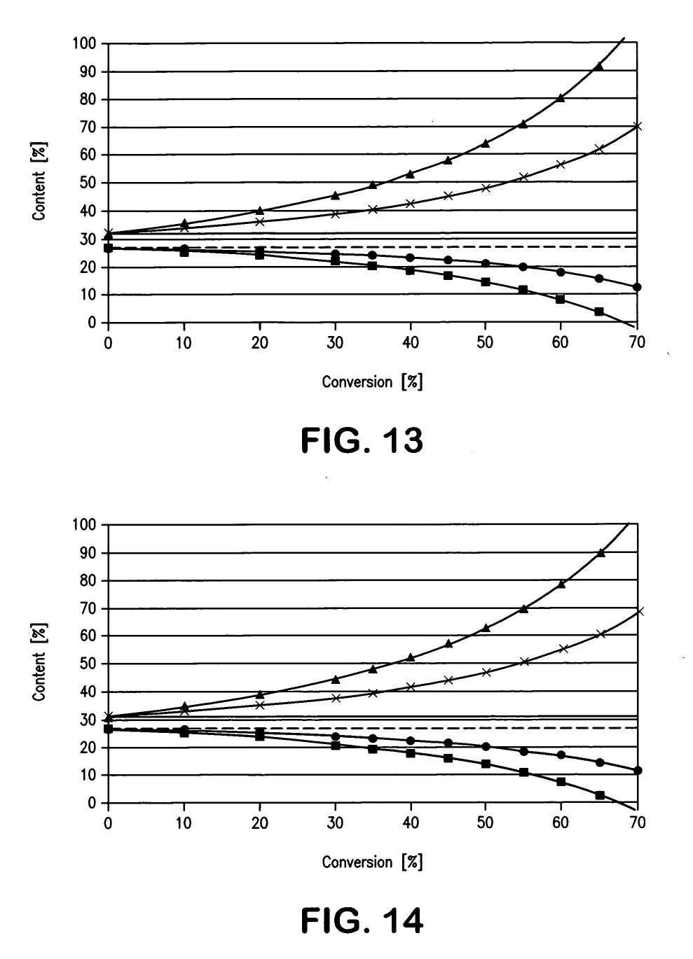

FIG. 11: graphic representation of the maximal and semi-maximal contents of 5×- and 6×-unsaturated fatty acids and the corresponding contents of saturated fatty acids bound in the glyceride fraction for a conversion of 0 to 70% calculated for the fish oil Napro 18/12.
FIG. 12: graphic representation of the maximal and semi-maximal contents of 5×- and 6×-unsaturated fatty acids and the corresponding contents of saturated fatty acids bound in the glyceride fraction for a conversion of 0 to 70% calculated for the fish oil Napro 11/19.
FIG. 13: graphic representation of the maximal and semi-maximal contents of 5×- and 6×-unsaturated fatty acids and the corresponding contents of saturated fatty acids bound in the glyceride fraction for a conversion of 0 to 70% calculated for the fish oil Lamotte Type 170.
FIG. 14: graphic representation of the maximal and semi-maximal contents of 5×- and 6×-unsaturated fatty acids and the corresponding contents of saturated fatty acids bound in the glyceride fraction for a conversion of 0 to 70% calculated for the fish oil Brudy Algatrium DHA 18.

The following symbols are used in FIGS. 11 to 14:

▴=Maximal content of 5×- and 6×-unsaturated fatty acids in % as a function of the conversion
Solid line=content of 5×- and 6×-unsaturated fatty acids in % for a non-selective reaction
x=Semi-maximal enrichment of 5×- and 6×-unsaturated fatty acids in %
▪=Percentage content of saturated fatty acids for maximal enrichment of 5×- and 6×-unsaturated fatty acids in % Chain line=content of saturated fatty acids in % for a non-selective reaction
•=Percentage content of saturated fatty acids for semi-maximal enrichment of 5×- and 6×-unsaturated fatty acids in %

What we claim is:

1. A mixture comprising:
(A) optionally at least one monoglyceride corresponding to formula (I) or (II): 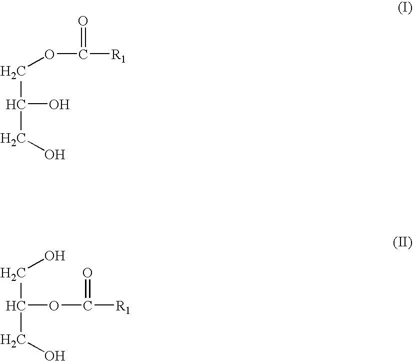
(B) optionally at least one diglyceride corresponding to formula (III) or (IV): 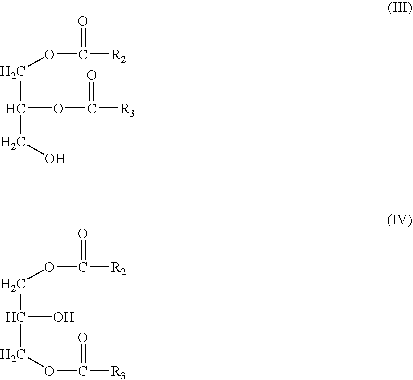
and
(C) at least one triglyceride corresponding to formula (V): 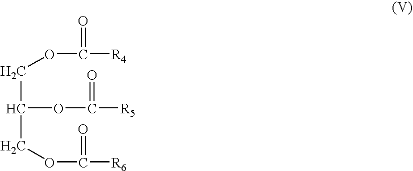
the groups R₁—CO—, R₂—CO—, R₃—CO—, R₄—CO—, R₅—CO— and R₆—CO— independently of one another being selected from the group consisting of a saturated fatty acid acyl group, a 1× to 4×-unsaturated fatty acid acyl group and an at least 5×-unsaturated fatty acid acyl group;
the sum of the weights of all at least 5×-unsaturated fatty acid acyl groups present in the mixture, expressed as free fatty acids, based on the sum of the weights of all fatty acid acyl groups present in the mixture, amounting to at least 40% by weight and at most 80% by weight;
and the sum of the weights of all saturated fatty acid acyl groups present in the mixture, expressed as free fatty acid, based on the sum of the weights of all fatty acid acyl groups present in the mixture, amounting to at most
$$\frac{85 - a}{3} \% by weight ,$$
α being the sum of the weights of all at least 5×-unsaturated fatty acid acyl groups present in the mixture, expressed as free fatty acids, based on the sum of all fatty acid acyl groups present in the mixture in % by weight;
and the weight of fatty acid acyl groups in stearic acid, expressed as free fatty acid, based on the sum of the weights of all fatty acid acyl groups present in the mixture, amounting to at most 2% by weight;
and the content of triglycerides, based on all the glycerides corresponding to formulae I to V, being from 50 to 85% by weight and the content of triglycerides, based on all the glycerides corresponding to formulae I to V, amounting to at least
$$\frac{410 - 5 \cdot a}{3} \% by weight ,$$
wherein α is as defined above,
and the content of diglycerides, based on all the glycerides corresponding to formulae I to V, being at least
100−3−b % by weight,
wherein b is the content of triglycerides, based on all the glycerides corresponding to formulae I to V, in % by weight.

2. A food supplement or an additive for a human or an animal food product which comprises the mixture of claim 1.

3. A process for the production of the mixture of claim 1 from a first mixture comprising:
(A) optionally at least one monoglyceride corresponding to formula (I) or (II): 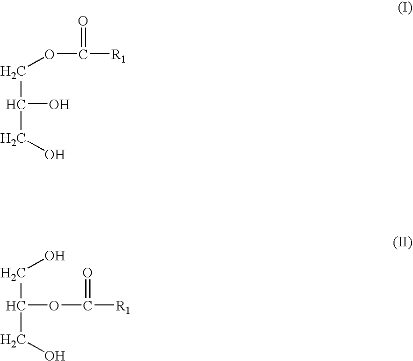
(B) optionally at least one diglyceride corresponding to formula (III) or (IV): 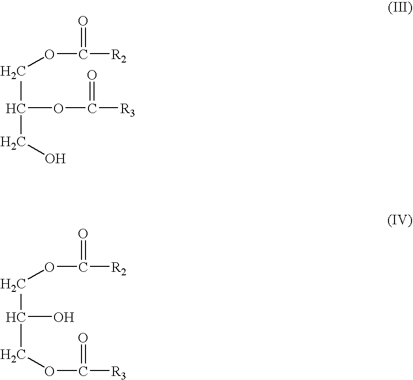
and
(C) at least one triglyceride corresponding to formula (V): 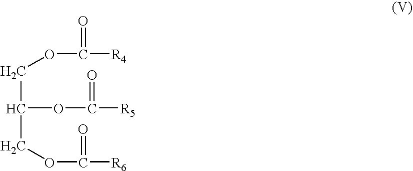
the groups R₁—CO—, R₂—CO—, R₃—CO—, R₄—CO—, R₅—CO— and R₆—CO— independently of one another being selected from the group consisting of a saturated fatty acid acyl group, a 1×- to 4×-unsaturated fatty acid acyl group and an at least 5×-unsaturated fatty acid acyl group;
and the sum of the weights of all at least 5×-unsaturated fatty acid acyl groups present in the mixture, expressed as free fatty acids, based on the sum of the weights of all fatty acid acyl groups present in the mixture, being higher than in the first mixture by a factor of at least 1.3 (factor A);
and the sum of the weights of all saturated fatty acid acyl groups present in the mixture, expressed as free fatty acid, based on the sum of the weights of all fatty acid acyl groups present in the mixture, being lower than in the first mixture by a factor of at least 2 (factor B);
and the value of factor A being smaller than the value of factor B;
and the content of triglycerides in the first mixture, based on all the glycerides corresponding to formula I to V, being at least 90% by weight;
and the first mixture containing at least 5×-unsaturated fatty acid acyl groups and the sum of the weights of all at least 5×-unsaturated fatty acid acyl groups present in the first mixture, expressed as free fatty acids, based on the sum of the weights of all fatty acid acyl groups present in the first mixture;
which process comprises reacting the first mixture with water or with a C₁₋₄ alcohol in the presence of a first, non-regioselective lipase having a high specificity for saturated fatty acids by comparison with all unsaturated fatty acids.

4. The process of claim 3, wherein the reaction of the first mixture with water or with a C₁₋₄ alcohol is carried out in the presence of a first, non-regioselective lipase having a high specificity for saturated fatty acids and a second, non-regioselective lipase having a high specificity for monounsaturated fatty acids by comparison with all unsaturated fatty acids.

5. The process of claim 3, wherein the first lipase is lipase A from Candida antarctica or an at least 80% structurally similar lipase.

6. The process of claim 4 wherein the second lipase is selected from one of the lipases from Candida rugosa or Candida cylindracea, or an at least 80% structurally similar lipase, or a mixture of said lipases thereof.

7. The process of claim 3 wherein the first mixture is selected from the group consisting of a fish oil, an oil from a marine crustaceans, an oil from microalgae, an oil from marine microorganisms, and an oil from marine mammals, said oil containing more than 20% by weight of at least 5×-unsaturated fatty acids.

8. The process of claim 3, wherein the first lipase is used in free or immobilized form.

9. The process of claim 8 wherein the first lipase is immobilized onto a nonionic polymer.

10. The process of claim 9 wherein the nonionic polymer is a polypropylene or polyacrylate-based carrier.

11. The process of claim 4 wherein the second lipase is used in free or immobilized form.

12. The process of claim 3 wherein water is added in an amount of 2 to 50% by weight of the mixture.

13. The process of claim 3 wherein a C₁₋₄ alcohol is added in an amount of from 5 to 50% by weight of the mixture.

14. The process of claim 13 wherein water and a C₁₋₄ alcohol mixture in an amount of from 5 to 50% by weight of the mixture is added.

15. The process of claim 13 wherein the alcohol is ethanol.
